SEQUENCE LISTING (23 sequences)

SEQ ID NO: 1 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 2 - DNA, 21 bp, synthetic construct
  source 1..21: mol_type = other DNA
ggttcctggaatgcagctctt

SEQ ID NO: 3 - DNA, 21 bp, synthetic construct
  source 1..21: mol_type = other DNA
tgccaccattctttcttctgc

SEQ ID NO: 4 - DNA, 21 bp, synthetic construct
  source 1..21: mol_type = other DNA
ttcaggttcctggaatgcagc

SEQ ID NO: 5 - DNA, 22 bp, synthetic construct
  source 1..22: mol_type = other DNA
acacaagtgggatcttcccaaa

SEQ ID NO: 6 - DNA, 21 bp, synthetic construct
  source 1..21: mol_type = other DNA
caggttcctggaatgcagctc

SEQ ID NO: 7 - DNA, 22 bp, synthetic construct
  source 1..22: mol_type = other DNA
ccacaaggaaatactggtgtgg

SEQ ID NO: 8 - DNA, 23 bp, synthetic construct
  source 1..23: mol_type = other DNA
gtaagttcaggttcctggaatgc

SEQ ID NO: 9 - DNA, 22 bp, synthetic construct
  source 1..22: mol_type = other DNA
cacaagtgggatcttcccaaaa

SEQ ID NO: 10 - DNA, 22 bp, synthetic construct
  source 1..22: mol_type = other DNA
taagttcaggttcctggaatgc

SEQ ID NO: 11 - DNA, 23 bp, synthetic construct
  source 1..23: mol_type = other DNA
aaagcagccacaaggaaatactg

SEQ ID NO: 12 - DNA, 22 bp, synthetic construct
  source 1..22: mol_type = other DNA
aggttcctggaatgcagctctt

SEQ ID NO: 13 - DNA, 23 bp, synthetic construct
  source 1..23: mol_type = other DNA
acacaagtgggatcttcccaaaa

SEQ ID NO: 14 - DNA, 22 bp, synthetic construct
  source 1..22: mol_type = other DNA
agttcatgcttggctattgagt

SEQ ID NO: 15 - DNA, 22 bp, synthetic construct
  source 1..22: mol_type = other DNA
tctgccaccattctttcttctg

SEQ ID NO: 16 - DNA, 22 bp, synthetic construct
  source 1..22: mol_type = other DNA
gttcaggttcctggaatgcagc

SEQ ID NO: 17 - DNA, 23 bp, synthetic construct
  source 1..23: mol_type = other DNA
aacacaagtgggatcttcccaaa

SEQ ID NO: 18 - DNA, 23 bp, synthetic construct
  source 1..23: mol_type = other DNA
ggttcctggaatgcagctctttt

SEQ ID NO: 19 - DNA, 23 bp, synthetic construct
  source 1..23: mol_type = other DNA
ctgaaacacaagtgggatcttcc

SEQ ID NO: 20 - DNA, 22 bp, synthetic construct
  source 1..22: mol_type = other DNA
tcaggttcctggaatgcagctc

SEQ ID NO: 21 - DNA, 23 bp, synthetic construct
  source 1..23: mol_type = other DNA
ccacaaggaaatactggtgtggt

SEQ ID NO: 22 - DNA, 21 bp, synthetic construct
  source 1..21: mol_type = other DNA
ggttcctggaatgcagctctt

SEQ ID NO: 23 - DNA, 21 bp, synthetic construct
  source 1..21: mol_type = other DNA
tgccaccattctttcttctgc

(19) United States
(12) Patent Application Publication
(10) Pub. No.: US 2025/0297315 A1
(43) Pub. Date: Sep. 25, 2025

(54) METHODS AND COMPOSITIONS FOR PROGNOSIS AND TREATMENT OF DILATED CARDIOMYOPATHY AND HEART FAILURE

(71) Applicants: THE BROAD INSTITUTE, INC., Cambridge, MA (US); THE GENERAL HOSPITAL CORPORATION, Boston, MA (US); THE UNITED STATES GOVERNMENT AS REPRESENTED BY THE DEPARTMENT OF VETERANS AFFAIRS, Washington, DC (US)

(72) Inventors: Krishna ARAGAM, Boston, MA (US); Jennifer HUFFMAN, Washington, DC (US); Liam GAZIANO, Cambridge, MA (US)

(73) Assignees: THE BROAD INSTITUTE, INC., Cambridge, MA (US); THE GENERAL HOSPITAL CORPORATION, Boston, MA (US); THE UNITED STATES GOVERNMENT AS REPRESENTED BY THE DEPARTMENT OF VETERANS AFFAIRS, Washington, DC (US)

(21) Appl. No.: 19/061,034

(22) Filed: Feb. 24, 2025

Related U.S. Application Data
(63) Continuation of application No. PCT/US2023/031136, filed on Aug. 25, 2023.
(60) Provisional application No. 63/401,343, filed on Aug. 26, 2022.

(51) Int. Cl. C12Q 1/6883; A61K 45/00; G01N 33/68

(52) U.S. Cl. CPC C12Q 1/6883; A61K 45/00; G01N 33/6893; C12Q 2600/106; C12Q 2600/158; G01N 2333/70596; G01N 2800/325; G01N 2800/52

(57) ABSTRACT

Provided herein are methods and compositions for the diagnosis, prognosis, and treatment of dilated cardiomyopathy (DCM) and heart failure (HF). In particular, provided are methods and compositions for detecting a loss-of-function mutation in the CD36 gene. Provided are methods of determining the likelihood that a subject will respond to a treatment for DCM or HF, based on the identification of a CD36 mutation in a sample from the subject.

### CROSS-REFERENCE TO RELATED APPLICATIONS

This application is a continuation of International Application No. PCT/US2023/031136, filed on Aug. 25, 2023, which claims the benefit under 35 U.S.C. § 119(e) of U.S. Provisional Patent Application No. 63/401,343, filed Aug. 26, 2022, the entire content of which are incorporated herein by reference in their entirety.

### STATEMENT OF U.S. GOVERNMENT SUPPORT

This invention was made with government support under Grant Nos. I01-CX001737 and I01-BX004821 awarded by the Department of Veterans Affairs Office of Research and Development, Million Veteran Program; Grant No. I01CX001922 awarded by the Department of Veterans Affairs; Grant No. K081HL153937 awarded by the National Institutes of Health/National Heart Lung and Blood Institute; and Grant No. 862032 awarded by the American Heart Association. The government has certain rights in the invention.

### SEQUENCE LISTING

The instant application contains a Sequence Listing which has been submitted electronically in XML file format and is hereby incorporated by reference in its entirety. Said XML copy, created on Jun. 9, 2025, is named 114203-10402_SL.xml and is 21,784 bytes in size.

### BACKGROUND

Individuals of self-identified Black race are at disproportionate risk for dilated cardiomyopathy (DCM), a preeminent cause of heart failure with reduced ejection fraction (HFrEF) and the most common indication for cardiac transplantation. An approximately two-fold odds of DCM has been reported for Black, as compared to White individuals, an observation not fully explained by differences in risk factor burden or socioeconomic factors such as access to care. An improved understanding of the factors that contribute to these disparate, race-specific, risk profiles is therefore critical to reduce the excess toll of DCM that afflicts the Black population.

A distinct genetic basis for DCM among Black individuals has been postulated, invoking the possibility of genetic polymorphisms specific to those of African ancestral groups. While many rare and common genetic variants have now been implicated in the pathogenesis of DCM, their identification has relied on the study of populations composed largely of individuals of European genetic ancestry. Investigations for DCM-associated polymorphisms of particular relevance to African ancestral groups have therefore been limited by the modest numbers of African genetic ancestry participants in most cohorts with genetic data.

There is a pressing need in the art for technologies for the prognosis, prevention, and treatment of DCM and HF, including the prognosis, prevention, and treatment of DCM and HF in certain patient populations, including individuals of African ancestry.

### SUMMARY OF THE INVENTION

The present disclosure provides technologies for the diagnosis, prognosis, prevention, and treatment of dilated cardiomyopathy (DCM) and heart failure. Provided are methods and compositions for detecting a variant nucleotide sequence in a CD36 gene in an individual.

In one aspect, provided is a method of detecting CD36 expression or function in a sample, comprising, consisting of, or consisting essentially of (a) obtaining a sample from a subject having, suspected of having, or at risk for dilated cardiomyopathy (DCM) or heart failure (HF); and (b) detecting (i) an expression level of full-length CD36 protein in the sample, or (ii) the presence or absence of a nucleic acid encoding a CD36 protein that comprises a mutation that results in a loss of function of the CD36 protein encoded by the nucleic acid. In some embodiments, detecting comprises sequencing the nucleic acid encoding the CD36 protein. In some embodiments, detecting comprises amplifying the nucleic acid encoding the CD36 protein.

In one aspect, provided is a method for prognosing a subject having, suspected of having, or at risk for dilated cardiomyopathy (DCM) or heart failure (HF), comprising: (a) detecting in a sample obtained from the subject an expression level of full-length CD36 protein; and (b) prognosing the subject as having a poor prognosis if the expression level of CD36 protein is less than a reference level, wherein the reference level is the corresponding level of expression of CD36 protein in a sample obtained from a subject not having or not suspected of having DCM or HF. In some embodiments, HF is heart failure with reduced ejection fraction (HFrEF).

In one aspect, provided is a method for determining whether a subject having, suspected of having, or at risk for dilated cardiomyopathy (DCM) or heart failure (HF) is likely to respond to a therapy for DCM or HF, comprising: (a) detecting in a sample obtained from the subject an expression level of full-length CD36 protein; and (b) determining that the subject is less likely to respond to the therapy if the expression level of CD36 protein is less than a reference level when compared to a subject whose CD36 protein expression level is not less than the reference level, wherein the reference level is the corresponding level of expression of CD36 protein in a sample obtained from a subject not having or not suspected of having DCM or HF. In some embodiments of the method, HF is heart failure with reduced ejection fraction (HFrEF).

In one aspect, provided is a method for determining whether a subject having, suspected of having, or at risk for dilated cardiomyopathy (DCM) or heart failure (HF) is likely to respond to a therapy for DCM or HF, comprising: (a) analyzing a biological sample obtained from the subject, wherein the biological sample comprises a CD36 protein or a nucleic acid encoding a CD36 protein in the sample; (b) detecting the presence or absence of a CD36 mutation that results in loss-of-function of CD36; and (c) determining that the subject is less likely to respond to the therapy if the mutation is detected. In some embodiments of the method, HF is heart failure with reduced ejection fraction (HFrEF). In some embodiments, the mutation is a stop-gain variant. In some embodiments, the mutation is Y325X relative to SEQ ID NO:1. In some embodiments, the therapy is selected from the group consisting of salt restriction, ACE inhibitors, diuretics, beta blockers, anticoagulants, coenzyme Q, angiotensin receptor blockers (ARBs), aldosterone antagonists, and sodium glucose cotransporter-2 (SGLT-2) inhibitors.

In one aspect, provided is a method for treating dilated cardiomyopathy (DCM) or heart failure (HF) in a subject, the method comprising: (a) analyzing a biological sample obtained from the subject, wherein the biological sample comprises a CD36 protein or a nucleic acid encoding a CD36 protein in the sample; (b) detecting the presence or absence of a CD36 mutation that results in loss-of-function of CD36; and (c) administering to the subject a pharmacological agent targeting myocardial energetics if the mutation is detected. In some embodiments, of the method, HF is heart failure with reduced ejection fraction (HFrEF). In some embodiments, the mutation is a stop-gain variant. In some embodiments, the mutation is Y325X relative to SEQ ID NO:1. In some embodiments, the method further comprises (d) administering to the subject a pharmacological agent selected from the group consisting of salt restriction, ACE inhibitors, diuretics, beta blockers, anticoagulants, and coenzyme Q, angiotensin receptor blockers (ARBs), aldosterone antagonists, and sodium glucose cotransporter-2 (SGLT-2) inhibitors, if the mutation is not detected.

In one aspect, provided is a method for treating dilated cardiomyopathy (DCM) or heart failure (HF) in a subject, the method comprising: (a) analyzing a biological sample obtained from the subject, wherein the biological sample comprises a CD36 protein or a nucleic acid encoding a CD36 protein in the sample; (b) detecting in the biological sample an expression level of full-length CD36 protein; and (c) administering to the subject a pharmacological agent targeting myocardial energetics if the expression level of CD36 is less than a reference level, wherein the reference level is the corresponding level of expression of CD36 protein in a sample obtained from a subject not having or not suspected of having DCM or HF. In some embodiments of the method, HF is heart failure with reduced ejection fraction (HFrEF). In some embodiments, the method further comprises (e) administering to the subject a pharmacological agent selected from the group consisting of salt restriction, ACE inhibitors, diuretics, beta blockers, anticoagulants, and coenzyme Q, angiotensin receptor blockers (ARBs), aldosterone antagonists, and sodium glucose cotransporter-2 (SGLT-2) inhibitors, if the expression level of CD36 is not less than the reference level. In some embodiments, the pharmacological agent is a gene therapy.

In one aspect, provided is a kit for prognosing dilated cardiomyopathy (DCM) or heart failure (HF) in a patient diagnosed with DCM or HF comprising: (i) at least one PCR primer pair for PCR amplification of a CD36 gene or at least one probe for hybridizing to a CD36 gene under stringent hybridization conditions; and (ii) at least one PCR primer pair for PCR amplification of at least one housekeeping gene. In some embodiments, the kit further comprises instructions for using the kit. In some embodiments, at least one primer of a PCR primer pair for PCR amplification of a CD36 gene hybridizes to a nucleic acid sequence encoding Y325X of SEQ ID NO:1. In some embodiments, the at least one housekeeping genes is selected from the group consisting of GAPDH, ACTB, TUBB, UBQ, PGK, and RPL. In some embodiments, the at least one PCR primer pair for PCR amplification of a CD36 gene is selected from the group consisting of primer pair #1 (SEQ ID NO:2-3), primer pair #2 (SEQ ID NO:4-5), primer pair #3 (SEQ ID NO:6-7), primer pair #4 (SEQ ID NO:8-9), primer pair #5 (SEQ ID NO:10-11), primer pair #6 (SEQ ID NO:12-13), primer pair #7 (SEQ ID NO:14-15), primer pair #8 (SEQ ID NO:16-17), primer pair #9 (SEQ ID NO:18-19), primer pair #10 (SEQ ID NO:20-21), and primer pair #11 (SEQ ID NO:22-23).

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows a diagram summarizing the various analyses performed in the study detailed herein.

FIG. 2A shows a Manhattan plot showing association between genetic variants and dilated cardiomyopathy across the genome.

FIG. 2B shows an inset table showing the minor allele frequency for the functional variant (rs3211938) across global populations as found in dbSNP (ncbi.nlm.nih.gov/snp/).

FIG. 2C shows a regional plot showing the associations at the CD36 locus as well as the linkage disequilibrium with the lead SNP (rs3211916) based on 1000Genomes AFR population.

FIG. 3A shows replication of association between rs3211938 and phenotypes relating to left ventricular dysfunction. Shown are effects for the heterozygous genotype (T/G) and homozygous risk genotype (G/G) versus the referent genotype (T/T). Associations for DCM were estimated using logistic regression adjusting for age, sex, and the first 10 principal components in African ancestry individuals of the Million Veteran Program (MVP) and Penn Medicine Biobank (PMBB).

FIG. 3B shows associations for HFrEF estimated using the methods described above for (A).

FIG. 3C shows associations for cardiac MRI-derived left ventricular traits estimated using linear regression adjusting for age, sex, and the first 10 principal components in African ancestry individuals from the UK Biobank (UKB), the Multi-Ethnic Study of Atherosclerosis (MESA), the Jackson Heart Study (JHS). LVEF=left ventricular ejection fraction; LVESVi=left ventricular end-systolic volume indexed for body surface area; LVEDVi=left ventricular end-diastolic volume indexed for body surface area; LVMi=left ventricular mass indexed for body surface area.

FIG. 4A shows associations of rs3211938 with a phenome-wide range of binary outcomes and traits, in particular associations for PheCodes tested using logistic regression and adjusted for age, sex, and 10 principal components in up to 120,911 AFR ancestry members of the Million Veteran Program. A total of 1,808 PheCodes were tested, and results that fell below Bonferroni-corrected significance threshold (2.77×10⁻⁵) are shown.

FIG. 4B shows estimates for continuous traits, which were tested using linear regression and adjusted for age, sex, and 10 principal components in AFR ancestry members of the Million Veteran Program or UK Biobank. Traits were scaled to have a standard deviation of one. A total of 247 traits were tested, and results meeting the Bonferroni-corrected significance threshold (2.02×10⁻⁰⁴) are shown. Estimates calculated in the UK Biobank population; all other results are from the Million Veteran Program. All analyses used an additive model with the G allele at rs3211938 serving as the effect allele.

FIG. 5A shows a graph illustrating ancestry-specific population attributable fractions for risk factors of dilated cardiomyopathy. Population attributable fraction (PAF) of clinical factors and rs3211938 for DCM are shown among participants of the Million Veteran Program of African ancestry (AFR).

FIG. 5B shows a graph illustrating ancestry-specific population attributable fractions for risk factors of dilated cardiomyopathy. Population attributable fraction (PAF) of clinical factors and rs3211938 for DCM are shown among participants of the Million Veteran Program of European ancestry (EUR). PAF values were truncated at a lower limit of zero, the lowest theoretical level of risk attributable to a factor. BMI=body mass index; HDL=high-density lipoprotein cholesterol.

FIG. 6A shows a graph illustrating the effect of CD36 siRNA on CD36 expression in iPSC-CMs as measured by PCR. The Y-axis displays the relative expression compared to that measured following administration of a control (scrambled) CD36 siRNA to the iPSC-CMs.

FIG. 6B shows a graph illustrating the effect of reduced CD36 expression (via administration of a CD36 siRNA) on lipid uptake over time in iPSC-CMs. The Y-axis displays the fluorescence normalized to protein.

FIG. 7A shows a graph illustrating the effect of reduced CD36 expression (via administration of a CD36 siRNA) on the FCCP-induced mitochondrial oxygen consumption rate (OCR) in iPSC-CMs. The Y-axis displays the OCR (pmol/min/mg protein).

FIG. 7B shows a graph illustrating the effect of reduced CD36 expression (via administration of a CD36 siRNA) on the maximal respiratory capacity in iPSC-CMs. The Y-axis displays the maximal respiratory capacity normalized to control (scrambled siRNA administration).

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### I. Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes instances where the event or circumstance occurs and instances where it does not.

A primer pair that specifically hybridizes under stringent conditions to a target nucleic acid may hybridize to any portion of the gene. As a result, the entire gene may be amplified or a segment of the gene may be amplified, depending on the portion of the gene to which the primers hybridize.

The terms “amplification” or “amplify” as used herein include methods for copying a target nucleic acid, thereby increasing the number of copies of a selected nucleic acid sequence. Amplification may be exponential or linear. A target nucleic acid may be DNA (such as, for example, genomic DNA and cDNA) or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, CA 1990, pp 13-20; Wharam, et al., Nucleic Acids Res. 2001 Jun. 1; 29(11):E54-E54; Hafner, et al., Biotechniques 2001 April; 30(4):852-860.

The terms “complement,” “complementary,” or “complementarity” as used herein with reference to polynucleotides (i.e., a sequence of nucleotides such as an oligonucleotide or a target nucleic acid) refer to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length. A nucleic acid that is the “full complement” or that is “fully complementary” to a reference sequence consists of a nucleotide sequence that is 100% complementary (under Watson/Crick pairing rules) to the reference sequence along the entire length of the nucleic acid that is the full complement. A full complement contains no mismatches to the reference sequence.

A “fragment” in the context of a nucleic acid refers to a sequence of nucleotide residues which are at least about 5 nucleotides, at least about 7 nucleotides, at least about 9 nucleotides, at least about 11 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention.

“Genomic nucleic acid” or “genomic DNA” refers to some or all of the DNA from a chromosome. Genomic DNA may be intact or fragmented (e.g., digested with restriction endonucleases by methods known in the art). In some embodiments, genomic DNA may include sequence from all or a portion of a single gene or from multiple genes. In contrast, the term “total genomic nucleic acid” is used herein to refer to the full complement of DNA contained in the genome. Methods of purifying DNA and/or RNA from a variety of samples are well-known in the art.

As used herein, the term “oligonucleotide” refers to a short polymer composed of deoxyribonucleotides, ribonucleotides or any combination thereof. Oligonucleotides are generally at least about 10, 11, 12, 13, 14, 15, 20, 25, 40 or 50 up to about 100, 110, 150 or 200 nucleotides (nt) in length, more preferably from about 10, 11, 12, 13, 14, or 15 up to about 70 or 85 nt, and most preferably from about 18 up to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target nucleic acid sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. An “exogenous primer” refers specifically to an oligonucleotide that is added to a reaction vessel containing the sample nucleic acid to be amplified from outside the vessel and is not produced from amplification in the reaction vessel. A primer that is “associated with” a fluorophore or other label is connected to label through some means. An example is a primer-probe.

Primers are typically from at least 10, 15, 18, or 30 nucleotides in length up to about 100, 110, 125, or 200 nucleotides in length, preferably from at least 15 up to about 60 nucleotides in length, and most preferably from at least 25 up to about 40 nucleotides in length. In some embodiments, primers and/or probes are 15 to 35 nucleotides in length. There is no standard length for optimal hybridization or polymerase chain reaction amplification. An optimal length for a particular primer application may be readily determined in the manner described in H. Erlich, PCR Technology, Principles and Application for DNA Amplification, (1989).

A “primer pair” is a pair of primers that are both directed to target nucleic acid sequence. A primer pair contains a forward primer and a reverse primer, each of which hybridizes under stringent condition to a different strand of a double-stranded target nucleic acid sequence. The forward primer is complementary to the anti-sense strand of the dsDNA and the reverse primer is complementary to the sense-strand. One primer of a primer pair may be a primer-probe (i.e., a bi-functional molecule that contains a PCR primer element covalently linked by a polymerase-blocking group to a probe element and, in addition, may contain a fluorophore that interacts with a quencher).

An oligonucleotide (e.g., a probe or a primer) that is specific for a target nucleic acid will “hybridize” to the target nucleic acid under specified conditions. As used herein, “hybridization” or “hybridizing” refers to the process by which an oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target nucleic acid hybridizes to a perfectly matched probe. Equations for calculating Tm and conditions for nucleic acid hybridization are known in the art. Specific hybridization preferably occurs under stringent conditions, which are well known in the art. Stringent hybridization conditions are hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

As used herein, an oligonucleotide is “specific” for a nucleic acid if the oligonucleotide has at least 50% sequence identity with the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

Oligonucleotides used as primers or probes for specifically amplifying (i.e., amplifying a particular target nucleic acid) or specifically detecting (i.e., detecting a particular target nucleic acid sequence) a target nucleic acid generally are capable of specifically hybridizing to the target nucleic acid under stringent conditions.

As used herein, the term “sample” or “test sample” may comprise clinical samples, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include nasopharyngeal swabs, wound swabs, and nasal washes. The term “patient sample” as used herein refers to a sample obtained from a human seeking diagnosis and/or treatment of a disease.

As used herein, the term “polymorphism” refers to the existence of two or more different nucleotide sequences at a particular locus in the DNA of the genome. Polymorphisms can serve as genetic markers and may also be referred to as genetic variants. Polymorphisms include nucleotide substitutions, insertions, deletions and microsatellites, and may, but need not, result in detectable differences in gene expression or protein function. A polymorphic site is a nucleotide position within a locus at which the nucleotide sequence varies from a reference sequence in at least one individual in a population.

“Haplotype,” as used herein, refers to a genetic variant or combination of variants carried on at least one chromosome in an individual. A haplotype often includes multiple contiguous polymorphic loci. All parts of a haplotype, as used herein, occur on the same copy of a chromosome or haploid DNA molecule. Absent evidence to the contrary, a haplotype is presumed to represent a combination of multiple loci that are likely to be transmitted together during meiosis. Each human carries a pair of haplotypes for any given genetic locus, consisting of sequences inherited on the homologous chromosomes from two parents. These haplotypes may be identical or may represent two different genetic variants for the given locus. Haplotyping is a process for determining one or more haplotypes in an individual. Haplotyping may include use of family pedigrees, molecular techniques and/or statistical inference.

A “variant” or “genetic variant” as used herein, refers to a specific isoform of a haplotype found in a population, the specific form differing from other forms of the same haplotype in at least one, and frequently more than one, variant sites or nucleotides within the region of interest in the gene. The sequences at these variant sites that differ between different alleles of a gene are termed “gene sequence variants,” “alleles,” or “variants.” The term “alternative form” refers to an allele that can be distinguished from other alleles by having at least one, and frequently more than one, variant sites within the gene sequence. “Variants” include isoforms having single nucleotide polymorphisms (SNPs) and deletion/insertion polymorphisms (DIPs). Reference to the presence of a variant means a particular variant, i.e., particular nucleotides at particular polymorphic sites, rather than just the presence of any variance in the gene.

The term “genotype” in the context of this invention refers to the particular allelic form of a gene, which can be defined by the particular nucleotide(s) present in a nucleic acid sequence at a particular site(s). Genotype may also indicate the pair of alleles present at one or more polymorphic loci. For diploid organisms, such as humans, two haplotypes make up a genotype. Genotyping is any process for determining a genotype of an individual, e.g., by nucleic acid amplification, DNA sequencing, antibody binding, or other chemical analysis (e.g., to determine the length). The resulting genotype may be unphased, meaning that the sequences found are not known to be derived from one parental chromosome or the other.

“Treat,” “treating,” or “treatment” as used herein refers to any type of measure that imparts a benefit to a patient afflicted with or at risk for developing a disease, including improvement in the condition of the patient (e.g., in one or more symptoms), delay in the onset or progression of the disease, prevention of disease, etc. Treatment may include any drug, drug product, method, procedure, lifestyle change, or other adjustment introduced in attempt to effect a change in a particular aspect of a subject's health (i.e., directed to a particular disease, disorder, or condition).

The term “pharmacological agent” or “therapeutic agent” as used herein refers to any composition that imparts a benefit to a subject or patient afflicted with or at risk for developing a disease, including improvement in the condition of the subject or patient (e.g., in one or more symptoms), delay in the onset or progression of the disease, prevention of disease, etc. A pharmacological agent or therapeutic agent may refer to a chemical compound, such as a drug, pro-drug, small-molecule drug, etc. A pharmacological agent or therapeutic agent may refer to a biological compound, such as a therapeutic nucleic acid, protein, peptide, polypeptide, protein complex, cell, cell extract, biological fluid, etc. A pharmacological agent or therapeutic agent can be or comprise a gene therapy. In some embodiments, a pharmacological agent includes a system for modulating the expression of one or more target genes. For example, a pharmacological agent can include a gene-editing or nuclease system, such as a Clustered Regularly Interspaced Short Palindromic Repeats (CRISPR)-Cas system.

The term “at risk”, as used in the context of a subject at risk for a particular disease or disorder (e.g., DCM or HF), refers to a likelihood that a subject will have or develop the particular disease. A subject may be at risk for a particular disease or disorder due to one or more factors. Factors may include but are not limited to genetic predispositions, age, height, weight, sex, race, nationality, ethnicity, sexual orientation, family health history, lifestyle and behavioral factors (such as diet, exercise, alcohol consumption, etc.), and clinical risk factors (e.g., other diseases or disorders). Generally, a subject at risk for a particular disease is a subject who does not have or who has not yet developed the particular disease. In some embodiments of the present disclosure, a subject may be at risk for developing DCM or HF due to their race (e.g., of African ancestry). Notable risk factors for DCM and HF include but are not limited to lifestyle and behavioral factors such as a high-sugar or high-fat diet, low exercise, and alcohol consumption, and clinical factors such as pre-existing atrial fibrillation, hypertension, coronary artery disease, obesity, and chronic kidney disease.

As used herein, the term “detecting” refers to observing a signal from a detectable label to indicate the presence of a target. More specifically, detecting is used in the context of detecting a specific sequence of a target nucleic acid molecule. The term “detecting” used in context of detecting a signal from a detectable label to indicate the presence of a target nucleic acid in the sample does not require the method to provide 100% sensitivity and/or 100% specificity. A sensitivity of at least 50% is preferred, although sensitivities of at least 60%, at least 70%, at least 80%, at least 90%, or at least 99% are more preferred. A specificity of at least 50% is preferred, although sensitivities of at least 60%, at least 70%, at least 80%, at least 90%, or at least 99% are more preferred. Detecting also encompasses assays that produce false positives and false negatives. False negative rates can be 1%, 5%, 10%, 15%, 20% or even higher. False positive rates can be 1%, 5%, 10%, 15%, 20% or even higher. As used herein, “detecting” may also refer to observing a signal indicating the presence and/or amount of a protein, such as a protein in a sample.

The terms “level,” “level of expression,” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (e.g., transfer and ribosomal RNAs).

In certain embodiments, the term “reference level” herein refers to a predetermined value. As the skilled artisan will appreciate, the reference level is predetermined and set to meet the requirements in terms of, for example, specificity and/or sensitivity. These requirements can vary, e.g., from regulatory body to regulatory body. It may be, for example, that assay sensitivity or specificity, respectively, has to be set to certain limits, e.g., 80%, 90%, or 95%. These requirements may also be defined in terms of positive or negative predictive values. Nonetheless, based on the teaching given in the present invention it will always be possible to arrive at the reference level meeting those requirements. In some embodiments, the reference level is determined in healthy individuals. The reference value in some embodiments has been predetermined in the disease entity to which the patient belongs (e.g., DCM or HF). In certain embodiments, the reference level can be set to any percentage between, e.g., 25% and 75% of the overall distribution of the values in a disease entity investigated. In other embodiments, the reference level can be set to, for example, the median, tertiles, quartiles, or quintiles as determined from the overall distribution of the values in a disease entity investigated or in a given population. In one embodiment, the reference level is set to the median value as determined from the overall distribution of the values in a disease entity investigated. In one embodiment, the reference level may depend on the gender of the patient, e.g., males and females may have different reference levels.

In certain embodiments, the term “at a reference level” refers to a level of a marker (e.g., full-length CD36 protein) that is the same as the level, detected by the methods described herein, from a reference sample.

In certain embodiments, the term “increase” or “above” refers to a level at the reference level or to an overall increase of 5%, 10%, 20%, 25%, 30%, 40%, 50%, 60%, 70%, 80%, 85%, 90%, 95%, 100%, or greater, in the level of a marker (e.g., full-length CD36 protein) detected by the methods described herein, as compared to the level from a reference sample.

In certain embodiments, the term “decrease” or “below” herein refers to a level below the reference level or to an overall reduction of 5%, 10%, 20%, 25%, 30%, 40%, 50%, 60%, 70%, 80%, 85%, 90%, 95%, 96%, 97%, 98%, 99% or greater, in the level of a marker (e.g., full-length CD36 protein) detected by the methods described herein, as compared to the level from a reference sample.

As used herein, the term “CD36 gene” refers to the CD36 gene, which encodes the CD36 protein. As used herein, the term can refer to any nucleic acid encoding a CD36 protein, such as genomic DNA, mRNA, cDNA, or other engineered/recombinant nucleic acid, or portions thereof. The term encompasses the nucleic acid sequences set forth in NCBI Accession Numbers NM_001001548.3, NM_001001547.3, NM_000072.3, NM_001127443.2, NM_001127444.2, NM_001289908.1, NM_001289909.1, NM_001289911.2, NR_110501.1, NM_001371074.1, NM_001371075.1, NM_001371077.1, NM_001371078.1, NM_001371079.1, NM_001371080.1, and NM_001371081.1, or the coding region thereof, as well as natural and engineered isoforms and variants. The term includes RNA transcripts encoding all or a portion of SEQ ID NO: 1, genomic sequences encoding SEQ ID NO: 1, and all untranslated CD36 genomic sequences, such as, for example, introns, untranslated leader regions, and polyadenylation signals. Illustrative nucleic acid sequences encompassed by the term are publicly available at National Center for Biotechnology Information, Bethesda, MD (www.ncbi.nlm.nih.gov) and HUGO Gene Nomenclature Committee, Cambridge, UK (www.genenames.org).

As used herein, the term “CD36 protein” refers generally to the CD36 protein, also known in the art as FAT; GP4; GP3B; GPIV; CHDS7; PASIV; SCARB3; BDPLT10. As used herein, the term can refer to any CD36 protein, polypeptide, or a portion thereof. The term encompasses the amino acid sequences set forth in NCBI Accession Numbers NP_001001548.1, NP_000063.2, NP_001001547.1, NP_001120915.1, NP_001120916.1, NP_001276837.1, NP_001276838.1, NP_001276840.1, NP_001358003.1, NP_001358004.1, NP_001358006.1, NP_001358007.1, NP_001358008.1, NP_001358009.1, and NP_001358010.1, as well as natural and engineered isoforms and variants. An amino acid sequence of human CD36 protein is set forth in SEQ ID NO:1.

(SEQ ID NO: 1)
MGCDRNCGLIAGAVIGAVLAVFGGILMPVGDLLIQKTIKKQVVLEEGTIA
FKNWVKTGTEVYRQFWIFDVQNPQEVMMNSSNIQVKQRGPYTYRVRFLAK
ENVTQDAEDNTVSFLQPNGAIFEPSLSVGTEADNFTVLNLAVAAASHIYQ
NQFVQMILNSLINKSKSSMFQVRTLRELLWGYRDPFLSLVPYPVTTTVGL
FYPYNNTADGVYKVFNGKDNISKVAIIDTYKGKRNLSYWESHCDMINGTD
AASFPPFVEKSQVLQFFSSDICRSIYAVFESDVNLKGIPVYRFVLPSKAF
ASPVENPDNYCFCTEKIISKNCTSYGVLDISKCKEGRPVYISLPHFLYAS
PDVSEPIDGLNPNEEEHRTYLDIEPITGFTLQFAKRLQVNLLVKPSEKIQ
VLKNLKRNYIVPILWLNETGTIGDEKANMFRSQVTGKINLLGLIEMILLS
VGVVMFVAFMISYCACRSKTIK

As used herein, the term “CD36 mutant” or “CD36 variant” refers generally to a CD36 nucleic acid or an amino acid sequence that differs from the wild-type sequence of CD36, such as that set forth, for example, in SEQ ID NO: 1. The term includes all manner of mutations known in the art, including, but not limited to, insertions, deletions, substitutions, and inversions, encompasses both silent mutations and those that alter CD36 function, and encompasses gain-of-function and loss-of-function mutations. In some embodiments described herein, CD36 mutations comprise a single nucleotide polymorphism (SNP). In some embodiments, the SNP is rs3211938, which is a single-nucleotide variation (SNV) of T to G, which results in the introduction of a premature stop codon at amino acid position 325 of SEQ ID NO:1. rs3211938 is located at position chr7:80671133 (GRCh38.p13). Specifically, the SNP rs3211938 results in a change of an encoded Tyrosine (Y) to a stop codon. This change can be described as Y325X, Y325*, or Tyr325Ter.

CD36 nucleic acid and protein sequences described herein can be isolated from any source, including, but not limited to, a human patient, a laboratory animal or veterinary animal (e.g., dog, pig, cow, horse, rat, mouse, etc.), a sample therefrom (e.g. tissue or body fluid, or extract thereof), or a cell therefrom (e.g., primary cell or cell line, or extract thereof).

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

### II. Dilated Cardiomyopathy (DCM) and Heart Failure

Dilated cardiomyopathy (DCM) is a condition in which the heart becomes enlarged and cannot pump blood effectively. The progression of heart failure is associated with left ventricular (LV) remodeling, which manifests as gradual increases in left ventricular end-diastolic and end-systolic volumes, wall thinning, and a change in chamber geometry to a more spherical, less elongated shape. This process is usually associated with a continuous decline in ejection fraction. Accordingly, heart failure associated with DCM is commonly referred to as heart failure with reduced ejection fraction (HFrEF).

Hallmarks of DCM include generalized enlargement of the heart as seen upon normal chest X-ray. Pleural effusion may also be noticed, which is due to pulmonary venous hypertension. The electrocardiogram often shows sinus tachycardia or atrial fibrillation, ventricular arrhythmias, left atrial enlargement, and sometimes intraventricular conduction defects and low voltage. When left bundle-branch block (LBBB) is accompanied by right axis deviation (RAD), the rare combination is considered to be highly suggestive of dilated or congestive cardiomyopathy. Echocardiogram shows left ventricular dilatation with normal or thinned walls and reduced ejection fraction. Cardiac catheterization and coronary angiography are often performed to exclude ischemic heart disease. Cardiac magnetic resonance imaging (cardiac MRI) may also provide helpful diagnostic information in patients with dilated cardiomyopathy.

Traditional therapies used to treat DCM include pharmacological interventions. Such drug-related therapies can slow down progression and in some cases even improve the heart condition. Traditional pharmacological therapies include but are not limited to salt restriction, ACE inhibitors, diuretics, and beta blockers. Anticoagulants may also be used for antithrombotic therapy. There is some evidence for the benefits of coenzyme Q10 in treating heart failure.

DCM is more common in people of African ancestry than in people of Caucasian ancestry. However, DCM can occur in people of any race or ethnicity.

### III. CD36

CD36 is a scavenger receptor implicated in the sequestration of Plasmodium falciparum-infected red blood cells. Polymorphisms inducing CD36 loss-of-function have been associated with reduced Plasmodium falciparum: infectivity and protection against malaria, suggesting positive selection for such polymorphisms (including the SNV identified herein, rs3211938) in malaria-endemic regions such as Sub-Saharan African despite its deleterious effects on the myocardium.

CD36 is a membrane glycoprotein present on platelets, mononuclear phagocytes, adipocytes, hepatocytes, myocytes, and some epithelia. On microvascular endothelial cells, CD36 is a receptor for thrombospondin-1 and related proteins and functions as a negative regulator of angiogenesis. On phagocytes, through its functions as a scavenger receptor recognizing specific oxidized phospholipids and lipoproteins, CD36 participates in internalization of apoptotic cells, certain bacterial and fungal pathogens, and modified low-density lipoproteins, thus contributing to inflammatory responses and atherothrombotic diseases.

CD36 also binds long-chain fatty acids and facilitates their transport into cells, thus participating in muscle lipid utilization, adipose energy storage, and gut fat absorption, and possibly contributing to the pathogenesis of metabolic disorders, such as diabetes and obesity. On sensory cells, CD36 is involved in insect pheromone signaling and rodent fatty food preference. The signaling pathways downstream of CD36 involve ligand-dependent recruitment and activation of nonreceptor tyrosine kinases, specific mitogen-activated protein kinases, and the Vav family of guanine nucleotide exchange factors; modulation of focal adhesion constituents; and generation of intracellular reactive oxygen species. CD36 in many cells is localized in specialized cholesterol-rich membrane microdomains and may also interact with other membrane receptors, such as tetraspanins and integrins. Identification of the precise CD36 signaling pathways in specific cells elicited in response to specific ligands may yield novel targets for drug development. The function of CD36 is thoroughly reviewed in Silverstein & Febbraio, 2009, CD36, a Scavenger Receptor Involved in Immunity, Metabolism, Angiogenesis, and Behavior, Sci Signal., 2(72), which is incorporated herein by reference in its entirety.

### IV. Methods of detecting a CD36 variant

### Sample Collection and Preparation

The methods and compositions of the present invention can be used to detect mutations in the CD36 gene and other mutations described herein using a biological sample obtained from an individual (e.g., a human individual, patient, or subject). A sample can be obtained from a subject suspected of having a mutated nucleic acid sequence, for example, from a tissue or a fluid sample from the subject. The methods provided can be performed using any sample containing nucleic acid. In some embodiments, the nucleic acid is deoxyribonucleic acid (DNA). In some embodiments, the nucleic acid is ribonucleic acid (RNA). The sample can be processed to release or otherwise make available a nucleic acid for detection as described herein. The nucleic acid (e.g., DNA or RNA) can be isolated from the sample according to any methods well-known to those of skill in the art. Such processing can include steps of nucleic acid manipulation, e.g., preparing a cDNA by reverse transcription of RNA from the biological sample. Thus, the nucleic acid to be assayed by the methods of the invention can be genomic DNA, cDNA, single stranded DNA or mRNA.

Examples of biological samples include tissue samples or any cell-containing or acellular bodily fluids. Biological samples can be obtained by standard procedures and can be used immediately or stored, under conditions appropriate for the type of biological sample, for later use.

Methods of obtaining test samples are well-known to those of skill in the art and include, but are not limited to, aspirations, tissue sections, drawing of blood or other fluids, surgical or needle biopsies, and the like. The test sample can be obtained from an individual or patient diagnosed as having a cardiovascular disorder or suspected being afflicted with a cardiovascular disorder. In some embodiments, the test sample is obtained from an individual or patient that has received one or more treatments for a cardiovascular disorder. The test sample can be a cell-containing liquid or a tissue. Samples can include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples can also be processed, such as sectioning of tissues, fractionation, purification, or cellular organelle separation.

If necessary, the sample can be collected or concentrated by centrifugation and the like. The cells of the sample can be subjected to lysis, such as by treatments with enzymes, heat, surfactants, ultrasonication, or a combination thereof. The lysis treatment is performed in order to obtain a sufficient amount of nucleic acid derived from the individual's cells to detect using a nucleic acid detection assay, e.g. a detection assay using PCR.

Methods of plasma and serum preparation are well-known in the art. Either “fresh” blood plasma or serum, or frozen (stored) and subsequently thawed plasma or serum can be used. Frozen (stored) plasma or serum should optimally be maintained at storage conditions of −20° C. to −70° C. until thawed and used. “Fresh” plasma or serum can be refrigerated or maintained on ice until used, with nucleic acid (e.g., RNA, DNA or total nucleic acid) extraction being performed as soon as possible.

### Nucleic Acid Extraction and Amplification

The nucleic acid to be assayed can be assayed directly from a biological sample or extracted from the biological sample prior to detection. As described herein, the biological sample can be any sample that contains a nucleic acid molecule, such as a fluid sample, a tissue sample, or a cell sample. The biological sample can be from a subject which includes any animal, preferably a mammal. A preferred subject is a human, which can be a patient presenting to a medical provider for diagnosis or treatment of a disease. The volume of plasma or serum used in the extraction can be varied dependent upon clinical intent, but volumes of 100 μL to one milliliter of plasma or serum are usually sufficient.

Various methods of extraction are suitable for isolating the DNA or RNA. In general, the aim is to separate DNA present in the nucleus of the cell from other cellular components. The isolation of nucleic acid usually involves lysis of tissue or cells. This process is essential for the destruction of protein structures and allows for release of nucleic acids from the nucleus. Lysis is typically carried out in a salt solution, containing detergents to denature proteins or proteases (enzymes digesting proteins), such as Proteinase K, or in some cases both. It results in the breakdown of cells and dissolving of membranes. Methods of DNA isolation include, but are not limited to, phenol:chloroform extraction, high salt precipitation, alkaline denaturation, ion exchange column chromatography, resin binding, and paramagnetic bead binding. See, e.g. Maniatis et al., Molecular Cloning, A Laboratory Manual, 2d, Cold Spring Harbor Laboratory Press, page 16.54 (1989). Numerous commercial kits that yield suitable DNA and RNA include, but are not limited to, QIAamp™ mini blood kit, Agencourt Genfind™, Roche Cobas®, Roche MagNA Pure®, or phenol:chloroform extraction using Eppendorf Phase Lock Gels®, and the NucliSens extraction kit (Biomerieux, Marcy l'Etoile, France).

Nucleic acid extracted from tissues, cells, plasma or serum can be amplified using nucleic acid amplification techniques well-known in the art. Many of these amplification methods can also be used to detect the presence of mutations simply by designing oligonucleotide primers or probes to interact with or hybridize to a particular target sequence in a specific manner (e.g., allele specific primers and/or probes or primers that flank target nucleic acids sequences). By way of example, but not by way of limitation, these techniques can include, but are not limited to, polymerase chain reaction (PCR), reverse transcriptase polymerase chain reaction (RT-PCR), real-time PCR (qPCR), nested PCR, ligase chain reaction (LCA) (see Abravaya, K., et al., Nucleic Acids Research, 23:675-682, (1995)), branched DNA signal amplification (Urdea, M. S., et al., AIDS, 7 (suppl 2):S11-S14, (1993)), amplifiable RNA reporters, Q-beta replication, transcription-based amplification system (TAS), boomerang DNA amplification, strand displacement activation (SDA), cycling probe technology, isothermal nucleic acid sequence based amplification (NASBA) (see Kievits, T. et al., J Virological Methods, 35:273-286, (1991)), Invader Technology, helicase dependent amplification (HDA) Amplification Refractory Mutation System (ARMS), and other sequence replication assays or signal amplification assays. Methods of amplification are well-known in the art.

A variety of amplification enzymes are well-known in the art and include, for example, DNA polymerase, RNA polymerase, reverse transcriptase, Q-beta replicase, thermostable DNA and RNA polymerases. Because these and other amplification reactions are catalyzed by enzymes, in a single step assay the nucleic acid releasing reagents and the detection reagents should not be potential inhibitors of amplification enzymes if the ultimate detection is to be amplification based.

PCR is a technique for exponentially making numerous copies of a specific template DNA sequence. The reaction consists of multiple amplification cycles (i.e. thermocycling) and is initiated using a pair of primer sequences that hybridize to the 5′ and 3′ ends of the sequence to be copied. The amplification cycle typically includes an initial denaturation (i.e. strand separation) of the target nucleic acid, typically at about 95° C., followed by up to 50 cycles or more of (1) denaturation, (2) annealing the primers to the target nucleic acid at a temperature determined by the melting point (Tm) of the region of homology between the primer and the target, and (3) extension at a temperature dependent on the polymerase, most commonly 72° C. An extended period of extension is typically performed at the end of the cycling. In each cycle of the reaction, the DNA sequence between the primers is copied. Primers can bind to the copied DNA as well as the original template sequence, so the total number of copies increases exponentially with time. PCR can be performed as according to Whelan, et al., J of Clin Micro, 33(3):556-561 (1995). An exemplary PCR reaction mixture includes two specific primers, dNTPs, approximately 0.25 U of thermostable polymerase, such as a Taq polymerase, and 1×PCR Buffer, typically containing a buffer (e.g. Tris), a salt (e.g. KCl) and magnesium (MgCl2). The Tm of a primer varies according to the length, G+C content, and the buffer conditions, among other factors. As used herein, Tm refers to that in the buffer used for the reaction of interest.

### Detection of Variant Sequences

Variant nucleic acids can be amplified prior to detection or can be detected directly during an amplification step (i.e., “real-time” methods). In some embodiments, the target sequence is amplified and the resulting amplicon is detected by electrophoresis. In some embodiments, the specific mutation or variant is detected by sequencing the amplified nucleic acid, for example, Sanger sequencing or Next Generation Sequencing (NGS). Next-generation sequencing lowers the costs and greatly increases the speed over the industry standard dyeterminator methods. Examples of NGS include, but are not limited to, Massively Parallel Signature Sequencing (MPSS), Polony sequencing combined an in vitro paired-tag library with emulsion PCR, 454 pyrosequencing, Solexa sequencing, SOLiD technology, DNA nanoball, Heliscope single molecule, Single molecule real time (SMRT) and ion semiconductor sequencing.

In some embodiments, the target sequence is amplified using a labeled primer such that the resulting amplicon is detectably labeled. In some embodiments, the primer is fluorescently labeled. In some embodiments, at least one allele-specific primer is used (e.g. a primer the spans the deletion breakpoint site, i.e., spans the junction formed by the 5′ and 3′ ends of the deletion).

In some embodiments, PCR amplification is performed in order to amplify a CD36 gene, or variant, fragment, or exon thereof. In some embodiments, an exon comprising a putative variant (e.g., a nonsense variant described herein, e.g., rs3211938) is amplified. Exemplary primer sequences that can be used to amplify a CD36 exon comprising rs3211938 are shown in Table 1 below.

TABLE 1
Exemplary Primer Sequences for Amplification
of CD36

| SEQ | | | |
|---|---|---|---|
| ID | Primer | | |
| NO: | Pair | Direction | Nucleotide Sequence |
| 2 | #1 | Forward | GGTTCCTGGAATGCAGCTCTT |
| 3 | #1 | Reverse | TGCCACCATTCTTTCTTCTGC |
| 4 | #2 | Forward | TTCAGGTTCCTGGAATGCAGC |
| 5 | #2 | Reverse | ACACAAGTGGGATCTTCCCAAA |
| 6 | #3 | Forward | CAGGTTCCTGGAATGCAGCTC |
| 7 | #3 | Reverse | CCACAAGGAAATACTGGTGTGG |
| 8 | #4 | Forward | GTAAGTTCAGGTTCCTGGAATGC |
| 9 | #4 | Reverse | CACAAGTGGGATCTTCCCAAAA |
| 10 | #5 | Forward | TAAGTTCAGGTTCCTGGAATGC |
| 11 | #5 | Reverse | AAAGCAGCCACAAGGAAATACTG |
| 12 | #6 | Forward | AGGTTCCTGGAATGCAGCTCTT |
| 13 | #6 | Reverse | ACACAAGTGGGATCTTCCCAAAA |
| 14 | #7 | Forward | AGTTCATGCTTGGCTATTGAGT |
| 15 | #7 | Reverse | TCTGCCACCATTCTTTCTTCTG |
| 16 | #8 | Forward | GTTCAGGTTCCTGGAATGCAGC |
| 17 | #8 | Reverse | AACACAAGTGGGATCTTCCCAAA |
| 18 | #9 | Forward | GGTTCCTGGAATGCAGCTCTTTT |
| 19 | #9 | Reverse | CTGAAACACAAGTGGGATCTTCC |
| 20 | #10 | Forward | TCAGGTTCCTGGAATGCAGCTC |
| 21 | #10 | Reverse | CCACAAGGAAATACTGGTGTGGT |
| 22 | #11 | Forward | GGTTCCTGGAATGCAGCTCTT |
| 23 | #11 | Reverse | TGCCACCATTCTTTCTTCTGC |

For the methods provided herein, a single primer can be used for detection, for example as in single nucleotide primer extension or allele-specific detection of nucleic acid containing the mutation, or a second primer can be used which can be upstream or downstream of the allele-specific primer. One or more of the primers used can be allele-specific primers. Preferably, the allele-specific primer contains a portion of wild-type sequence, more preferably at least about 3-40 consecutive nucleotides of wild-type sequence.

In one embodiment, detection of a variant nucleic acid is performed using an RT-PCR assay, such as the TaqMan® assay, which is also known as the 5′ nuclease assay (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye can be at the 5′ most and the 3′ most ends, respectively or vice versa. Alternatively, the reporter dye can be at the 5′ or 3′ most end while the quencher dye is attached to an internal nucleotide, or vice versa. In yet another embodiment, both the reporter and the quencher can be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target mutation-containing template which is amplified during PCR, and the probe is designed to hybridize to the target mutation site only if a particular mutation allele (e.g., SNP, insertion or deletion) is present.

TaqMan® primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present invention are useful in diagnostic or prognostic assays for cardiovascular disorders and related pathologies, and can be readily incorporated into a kit format. The present invention also includes modifications of the TaqMan® assay well-known in the art such as the use of Molecular Beacon probes (U.S. Pat. Nos. 5,118,801 and 5,312,728) and other variant formats (U.S. Pat. Nos. 5,866,336 and 6,117,635).

Amplified fragments can be detected using standard gel electrophoresis methods. For example, in preferred embodiments, amplified fractions are separated on an agarose gel and stained with ethidium bromide by methods known in the art to detect amplified fragments.

In some embodiments, amplified nucleic acids are detected by hybridization with a mutation-specific probe. Probe oligonucleotides, complementary to a portion of the amplified target sequence can be used to detect amplified fragments. Amplified nucleic acids for each of the target sequences can be detected simultaneously (i.e., in the same reaction vessel) or individually (i.e., in separate reaction vessels). In some embodiments, the amplified DNA is detected simultaneously, using two distinguishably-labeled, gene-specific oligonucleotide probes, one which hybridizes to the first target sequence and one which hybridizes to the second target sequence. Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe can be labeled.

In some embodiments, two or more assays are performed for detection of any of the variants described herein. In some embodiments, the identity of any of the variants described herein is confirmed by nucleic acid sequencing. Strategies for detecting or measuring a variant nucleic acid are well known in the art.

### Detection of CD36 Protein

Several methods for detection of proteins are well-known in the art. Detection of the proteins can involve resolution of the proteins by SDS polyacrylamide gel electrophoresis (SDS-PAGE), followed by staining the proteins with suitable stain, for example, Coomassie Blue. The CD36 proteins with and without a mutation can be differentiated from each other and also from other proteins by Western blot analysis using mutation-specific antibodies. Methods for performing a Western blot are well-known in the art and described, for example, in W. Burnette W. N. Anal. Biochem. 1981; 112 (2): 195-203.

Alternatively, flow cytometry can be applied to detect the mutant and wild-type CD36 protein. Antibodies specific for either the mutant or wild-type protein can be coupled to beads and can be used in the flow cytometry analysis.

In some embodiments, protein microarrays can be applied to identify the various CD36 protein variants. Methods of protein arrays are well-known in the art. In one example, antibodies specific for each protein can be immobilized on the solid surface such as glass or nylon membrane. The proteins can then be immobilized on the solid surface through the binding of the specific antibodies. Antibodies can be applied that bind specifically to a second epitope (e.g., an epitope common to the mutant and wild-type) of the CD36 proteins. The first antibody/protein/second antibody complex can then be detected using a detectably labeled secondary antibody. The detectable label can be detected as provided herein for polynucleotides and as is known in the art.

In some embodiments, recombinant CD36 proteins can be engineered to contain the deletion mutation. In some embodiments, the recombinant CD36 proteins contain an epitope tag (e.g. a peptide tag, such as a myc or HA tag). In some embodiments, the epitope tag can be removed from the protein after expression and/or purification of the recombinant protein.

### V. Methods of Treating Dilated Cardiomyopathy and Heart Failure

### Methods of Treatment

The present disclosure provides methods of treating or preventing dilated cardiomyopathy (DCM) or heart failure (HF) in a subject having, suspected of having, or at risk for DCM or HF, the method comprising, consisting of, or consisting essentially of administering a therapy to the subject.

The present disclosure is the first to report a CD36 polymorphism that is able to predict and prognose DCM or HF, but also identify subjects with DCM or HF that may respond to certain treatments. Traditional treatments for DCM include, but are not limited to salt restriction, ACE inhibitors, angiotensin receptor blockers (ARBs), aldosterone antagonists, sodium glucose cotransporter-2 (SGLT-2) inhibitors, diuretics, and beta blockers. Anticoagulants may also be used in the setting of left ventricular thrombus. There is some evidence for the benefits of coenzyme Q10 in treating heart failure.

Provided herein is a method for preventing or treating clinical or subclinical dilated cardiomyopathy (DCM) or heart failure (HF) in a subject with or at risk for DCM/HF, the method comprising: (a) analyzing a biological sample obtained from the subject, wherein the biological sample comprises a CD36 protein or a nucleic acid encoding a CD36 protein in the sample; (b) detecting the presence or absence of a CD36 mutation that results in loss-of-function of CD36; and (c) if the mutation is detected, administering to the subject a pharmacological agent that targets mechanisms pertinent myocardial energetics to optimize myocardial substrate utilization.

Provided herein is a method for preventing or treating clinical or subclinical dilated cardiomyopathy (DCM) or heart failure (HF) in a subject, the method comprising: (a) analyzing a biological sample obtained from the subject, wherein the biological sample comprises a CD36 protein or a nucleic acid encoding a CD36 protein in the sample; (b) detecting in the biological sample an expression level of full-length CD36 protein; and (c) administering to the subject a pharmacological agent targeting myocardial energetic pathways if the expression level of CD36 is less than a reference level, wherein the reference level is the corresponding level of expression of CD36 protein in a sample obtained from a subject not having or not suspected of having DCM or HF. In some embodiments, the method further comprises (e) administering to the subject a therapy that does not directly target myocardial energetics if the expression level of CD36 is not less than the reference level. In some embodiments, a therapy that does not directly target myocardial energetics may be a therapy that specifically and/or directly enhances ventricular contractility, such as a myosin activator (e.g., omecamtiv mecarbil). Other non-limiting examples of a therapy that does not directly target myocardial energetics include salt restriction, ACE inhibitors, diuretics, beta blockers, anticoagulants, and coenzyme Q, angiotensin receptor blockers (ARBs), aldosterone antagonists, and sodium glucose cotransporter-2 (SGLT-2) inhibitors.

### Gene Therapy

Genetic variants in over 100 genes have been linked to DCM. A newly emerging DCM locus is at CD36 (cluster of differentiation 36, a.k.a. platelet glycoprotein 4, fatty acid translocase—“FAT, scavenger receptor class B member 3—SCARB3, glycoproteins 88—“GP88”, IIIB—“GPIIIB”, or IV—GPIV). Specifically, a common, CD36 loss-of-function mutation (rs3211938; p.Tyr325Ter) has been associated with increased risk of DCM as disclosed herein. Several observations suggest that this CD36 mutation is more prevalent in individuals of African genetic ancestry: 1) The CD36 genetic variant is most prevalent in isolated populations; and 2) The Genome Aggregation Database (gnomAD) shows marked allele frequency differences for the CD36 variant among individuals of African versus European ancestry. It is now hypothesized that deleterious variation at CD36 may contribute to the increased prevalence of DCM in individuals of African ancestry. As described in the Examples section below, it was found, for the first time, that a CD36 loss-of-function variant that is more prevalent in descendants of African ancestry may account for a sizeable proportion of the increased risk of DCM in this population, and may also provide a precise target for therapeutic intervention.

Accordingly, in certain embodiments, a method of diagnosing and treating a patient having or at risk for cardiac disease, comprises identifying in a patient sample, at least one CD36 genetic variant as compared to a control CD36 nucleic acid sequence, wherein detection of certain variants are predictive of whether an increase in CD36 levels is therapeutic for the patient, and, administering to the patient identified as having such a variant, a therapeutically effective amount of an agent wherein the agent modulates expression or amount of CD36 molecules, proteins or peptides thereof in a target cell or tissue, as compared to a normal control.

In certain embodiments, the genetic variant is a single nucleotide variant (SNV), in-frame insertion, deletions, substitutions or combinations thereof. In certain embodiments, the SNVs comprises rs3211938. In certain embodiments, the SNV results in the introduction of a stop codon. For example, the SNV may result in a change in a change of Y325X. Such a change may result in the production of a truncated protein product relative to a wild-type form of the protein.

In further embodiments, a method of treating a patient having or at risk for cardiac disease, wherein the patient has at least one CD36 nucleotide variant (NV) as compared to a control CD36 nucleic acid sequence, comprises administering to the patient a therapeutically effective amount of an agent wherein the agent modulates expression or amount of CD36 molecules, proteins or peptides thereof in a target cell or tissue.

In certain embodiments, provided is an agent for treating a patient having or at risk for DCM, or a patient having clinical or subclinical DCM, and identified as having a genetic variant in a CD36 gene, wherein detecting certain genetic variants is predictive of whether an increase in CD36 levels is therapeutic for the patient.

In certain embodiments, a therapeutic agent for treatment or prevention of diseases associated with CD36 and associated molecules and pathways thereof, in subjects, modulates the expression or amounts of CD36 in a cell. In some embodiments, compositions comprise nucleic acid sequences encoding CD36, including, without limitation, one or more of (i) cDNA, (ii) CD36-encoding RNA, mRNA, or chemically or structurally modified derivatives thereof (i.e., capped mRNAs, circular mRNAs, etc.), and (iii) sense and/or antisense sequences of CD36.

In certain embodiments, the agent comprises one or more gene-editing or nuclease systems to delete or edit the genetic variants in subjects wherein an increase in CD36 would not be therapeutic or may even be detrimental to the subject. In certain other embodiments, the gene-editing or nuclease system is used in conjunction with guide molecules to correct mutations that are detrimental to CD36 expression or activity.

Any suitable nuclease system can be used including, for example, clustered regularly interspaced short palindromic repeat (CRISPR) nucleases, Argonaute family of endonucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference. In certain embodiments, the system is an Argonaute nuclease system. In certain embodiments, the system is a CRISPR system.

In certain embodiments, the gene editing agent comprises a Clustered Regularly Interspaced Short Palindromic Repeat (CRISPR)-associated endonuclease/Cas (CRISPR/Cas).

In certain embodiments, the CRISPR/Cas comprises catalytically deficient Cas protein (dCas), orthologs, homologs, mutants variants or fragments thereof.

The compositions disclosed herein may include nucleic acids encoding a CRISPR-associated endonuclease, such as Cas9. In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

In embodiments, the CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The invention is by no means limited to these variants, and also encompasses all Cas9 variants (Slaymaker, I. M. et al. (2015)). The present invention also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems.

Embodiments of the invention also include a new type of class 2 CRISPR-Cas system found in the genomes of two bacteria recovered from groundwater and sediment samples. This system includes Cas1, Cas2, Cas4 and an approximately .about.980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas 1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2016 supra).

In some embodiments, a nucleic acid sequence of CD36 comprises at least about a 50% sequence identity to wild type CD36 or cDNA sequences thereof. In other embodiments, the CD36 nucleic acid sequence comprises at least about 75%, 80%, 85%, 90%, 95%, 96%, 97%, 98%, 99% sequence identity to wild type CD36 or cDNA sequences thereof.

In some embodiments, a nucleic acid sequence of CD36 further comprises one or more mutations, substitutions, deletions, variants or combinations thereof.

In some embodiments, the homology, sequence identity or complementarity, between a CD36 nucleic acid sequence comprising one or more mutations, substitutions, deletions, variants or combinations thereof and the native or wild type or cDNA sequences of CD36 is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In those cases where the variants detected in a sample are predictive that an increase of CD36 levels or activity would be therapeutic, an agent comprising a polynucleotide encoding CD36 molecules is administered to that subject. In one embodiment, an expression vector encodes a CD36 gene or cDNA sequences thereof, or modified sequences thereof. In one embodiment, the expression vector encodes a nucleic acid sequence comprising at least about 50% sequence identity to wild type CD36 or cDNA sequences thereof. In other embodiments, the nucleic acid sequence comprises at least about 75%, 80%, 85%, 90%, 95%, 96%, 97%, 98%, 99% sequence identity to wild type CD36 or cDNA sequences thereof.

Suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. In a particular example, the viral vector comprises a strong eukaryotic promoter operably linked to the polynucleotide e.g., a cytomegalovirus (CMV) promoter. In another example, the viral vector comprises a truncated base-editing system such as that described in Davis et al., Nature Biomedical Engineering, doi.org/10.1038/s41551-022-000911-4 that has been suitably modified to target and correct CD36 variants. In certain embodiments, the viral capsid has been modified for enhanced transduction of human cardiomyocytes.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992). 10.1351 Another delivery method is to use single stranded DNA producing vectors which can produce the CD36 intracellularly, for example, cardiac tissues. See for example, Chen et al, BioTechniques, 34: 167-171 (2003), which is incorporated herein, by reference, in its entirety.

Expression of CD36 may be controlled by any promoter/enhancer element known in the art, but these regulatory elements must be functional in the host selected for expression. In some embodiments, the promoter is a tissue specific promoter. Of particular interest are muscle specific promoters, and more particularly, cardiac specific promoters. These include the myosin light chain-2 promoter (Franz et al. (1994) Cardioscience, Vol. 5(4):235-43; Kelly et al. (1995) J. Cell Biol., Vol. 129(2):383-396), the alpha actin promoter (Moss et al. (1996) Biol. Chem., Vol. 271(49):31688-31694), the troponin 1 promoter (Bhaysar et al. (1996) Genomics, Vol. 35(1):11-23); the Na.sup.+/Ca.sup.2+ exchanger promoter (Barnes et al. (1997) J. Biol. Chem., Vol. 272(17):11510-11517), the dystrophin promoter (Kimura et al. (1997) Dev. Growth Differ., Vol. 39(3):257-265), the alpha7 integrin promoter (Ziober and Kramer (1996) J. Bio. Chem., Vol. 271(37):22915-22), the brain natriuretic peptide promoter (LaPointe et al. (1996) Hypertension, Vol. 27(3 Pt 2):715-22) and the alpha B-crystallin/small heat shock protein promoter (Gopal-Srivastava (1995) J. Mol. Cell. Biol., Vol. 15(12):7081-7090), alpha myosin heavy chain promoter (Yamauchi-Takihara et al. (1989) Proc. Natl. Acad. Sci. USA, Vol. 86(10):3504-3508) and the ANF promoter (LaPointe et al. (1988) J. Biol. Chem., Vol. 263(19):9075-9078).

Yeast expression systems can also be used according to the invention to express CD36. For example, the non-fusion pYES2 vector (XbaI, SphI, ShoI, NotI, GstXI, EcoRI, BstXI, BamH1, SacI, Kpn1, and HindIII cloning sites; Invitrogen) or the fusion pYESHisA, B, C (XbaI, SphI, ShoI, NotI, BstXI, EcoRI, BamH1, SacI, Kpn1, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

One exemplary delivery system is a recombinant viral vector that incorporates one or more of the polynucleotides therein, for example, about one polynucleotide. An exemplary viral vector used in the invention methods has a pfu (plague forming units) of from about 10.sup.8 to about 5.times.10.sup.10 pfu. In embodiments in which the polynucleotide is to be administered with a non-viral vector, use of between from about 0.1 nanograms to about 4000 micrograms will often be useful e.g., about 1 nanogram to about 100 micrograms.

In some embodiments, the vector is an adenovirus-associated viral vector (AAV), for example, AAV9. The term “AAV vector” means a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, such as the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype 1 (AAV 1), serotype 2 (AAV2), serotype 3 (AAV3), serotype 4 (AAV4), serotype 5 (AAVS), serotype 6 (AAV6), serotype 7 (AAV7), serotype 8 (AAV8), or serotype 9 (AAV9).

Some skilled in the art have circumvented some of the limitations of adenovirus-based vectors by using adenovirus “hybrid” viruses, which incorporate desirable features from adenovirus as well as from other types of viruses as a means of generating unique vectors with highly specialized properties. For example, viral vector chimeras were generated between adenovirus and adeno-associated virus (AAV). These aspects of the invention do not deviate from the scope of the invention described herein.

Nucleic acids encoding the CD36 proteins of the invention may be delivered to cardiac muscle by methods known in the art. For example, cardiac cells of a large mammal may be transfected by a method that includes dilating a blood vessel of the coronary circulation by administering a vasodilating substance to said mammal prior to, and/or concurrently with, administering the nucleic acids. In some embodiments, the method includes administering the nucleic acids into a blood vessel of the coronary circulation in vivo, wherein nucleic acids are infused into the blood vessel over a period of at least about three minutes, wherein the coronary circulation is not isolated or substantially isolated from the systemic circulation of the mammal, and wherein the nucleic acids transfect cardiac cells of the mammal.

In some embodiments, the subject can be a human, an experimental animal, e.g., a rat or a mouse, a domestic animal, e.g., a dog, cow, sheep, pig or horse, or a non-human primate, e.g., a monkey. The subject may be suffering from a cardiac disorder, such as heart failure, ischemia, myocardial infarction, congestive heart failure, arrhythmia, transplant rejection and the like. In one embodiment, the subject is suffering from heart failure. In another particular embodiment, the subject is suffering from arrhythmia. In one embodiment, the subject is a human. For example, the subject is between ages 18 and 65. In another embodiment, the subject is a non-human animal.

In one embodiment, the subject has or is at risk for heart failure, e.g. a non-ischemic cardiomyopathy, mitral valve regurgitation, ischemic cardiomyopathy, or aortic stenosis or regurgitation.

In some embodiments, transfection of cardiac cells with nucleic acid molecules encoding a CD36 protein or CD36 protein fused to an effector domain increases lateral ventricle fractional shortening. In some embodiments, the mammal is human and the disease is congestive heart failure. In some embodiments, the transfection of the cardiac cells increases lateral ventricle fractional shortening when measured about 4 months after said infusion by at least 25% as compared to lateral ventricle fractional shortening before infusion of the polynucleotide. In some embodiments, the transfection of the cardiac cells results in an improvement in a measure of cardiac function selected from the group consisting of expression of CD36 protein, fractional shortening, ejection fraction, cardiac output, time constant of ventricular relaxation, and regurgitant volume.

A treatment can be evaluated by assessing the effect of the treatment on a parameter related to contractility. For example, SR Ca.sup.2+ ATPase activity or intracellular Ca.sup.2+ concentration can be measured. Furthermore, force generation by hearts or heart tissue can be measured using methods described in Strauss et al., Am. J. Physiol., 262:1437-45, 1992, the contents of which are incorporated herein by reference.

Modified Nucleic Acid Sequences: It is not intended that the present invention be limited by the nature of the nucleic acid employed. The nucleic acid may be DNA or RNA and may exist in a double-stranded, single-stranded or partially double-stranded form.

Nucleic acids useful in the present invention include, by way of example and not limitation, oligonucleotides and polynucleotides such as antisense DNAs and/or RNAs; ribozymes; shRNAs; inhibitory nucleic acids; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach (IRL Press, Oxford, England)). RNAs may be produce in high yield via in vitro transcription using plasmids such as pGEM™ T vector or SP65 (Promega Corporation, Madison, Wis.).

Accordingly, certain nucleic acid sequences of this invention are chimeric nucleic acid sequences. “Chimeric nucleic acid sequences” or “chimeras,” in the context of this invention, contain two or more chemically distinct regions, each made up of at least one nucleotide. These sequences typically contain at least one region of modified nucleotides that confers one or more beneficial properties (such as, for example, increased nuclease resistance, increased uptake into cells, increased binding affinity for the target).

Chimeric nucleic acid sequences of the invention may be formed as composite structures of two or more oligonucleotides, modified oligonucleotides, oligonucleosides and/or oligonucleotide mimetics. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

Specific examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH.sub.2-NH—O—CH.sub.2, CH, —N(CH.sub.3)-O—CH.sub.2 [known as a methylene(methylimino) or MMI backbone], CH.sub.2-O—N(CH.sub.3)-CH.sub.2, CH.sub.2-N(CH.sub.3)-N(CH.sub.3)-CH.sub.2 and O—N(CH.sub.3)-CH.sub.2-CH.sub.2 backbones, wherein the native phosphodiester backbone is represented as O—P—O—CH). The amide backbones disclosed by De Mesmaeker et al. Ace. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

Exemplary modified oligonucleotide backbones comprise, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

The nucleic acid sequences may also include, additionally or alternatively, nucleobase (often referred to in the art simply as “base”) modifications or substitutions. As used herein, “unmodified” or “natural” nucleotides include adenine (A), guanine (G), thymine (T), cytosine (C) and uracil (U). Modified nucleotides include nucleotides found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleotides, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. (Kornberg, A., DNA Replication, W. H. Freeman & Co., San Francisco, 1980, pp 75-77; Gebeyehu, G., (1987) et al. Nucl. Acids Res. 15:4513). A “universal” base known in the art, e.g., inosine, may be included.

Another modification involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety, cholic acid, a thioether, e.g., hexyl-5-tritylthiol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid. Nucleic acid sequences comprising lipophilic moieties, and methods for preparing such oligonucleotides are known in the art, for example, U.S. Pat. Nos. 5,138,045, 5,218,105 and 5,459,255.

It is not necessary for all positions in a given nucleic acid sequence to be uniformly modified, and in fact more than one of the aforementioned modifications may be incorporated in a single nucleic acid sequence or even at within a single nucleoside within such sequences. The present invention also includes oligonucleotides which are chimeric oligonucleotides as hereinbefore defined.

In another embodiment, the CD36 nucleic acid molecule of the present invention is conjugated with another moiety including but not limited to basic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

In another embodiment, the CD36 nucleic acid sequences comprise one or more nucleotides substituted with locked nucleic acids (LNA). The LNA modified nucleic acid sequences may have a size similar to the parent or native sequence or may be larger or smaller. Such LNA-modified oligonucleotides may contain less than about 70%, or less than about 60%, or less than about 50% LNA monomers and that their sizes are between about 1 and 25 nucleotides.

### Subject Being Treated

A subject being treated for DCM or HF according to the disclosed methods and uses may exemplify one or more of the underlying gene expression patterns that are disclosed herein. In particular, a subject with or at risk for DCM or HF that is to be treated according to the disclosed methods and uses may express a polymorphism in the CD36 gene. In some embodiments, a subject with or at risk for DCM or HF may express a nonsense variant of CD36. In some preferred embodiments, a subject with or at risk for DCM or HF may express a loss-of-function variant of CD36. A subject may be heterozygous or homozygous for a CD36 variant. A CD36 polymorphism or variant may be observed in a sample or test sample obtained from a subject.

A subject may be a human individual of any race or gender. In some embodiments, a subject is a human individual with African ancestry.

Full-length CD36 protein may be underexpressed by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, or at least about 8.5-fold compared to the expression level in a sample from an individual that does not have DCM or HF. A CD36 gene encoding a full-length CD36 protein may be underexpressed by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, or at least about 8.5-fold compared to the expression level in a sample from an individual that does not have DCM or HF.

61| In some embodiments, the subject has been diagnosed with DCM or HF for at least about 1 month, at least about 2 months, at least about 3 months, at least about 4 months, at least about 5 months, or at least about 6 months.

In some embodiments, the subject has not been previously diagnosed with DCM or HF. In some embodiments, the subject does not have symptoms of DCM or HF.

### Doses and Dosing Regimen for the Disclosed Methods and Uses

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment and prevention dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

Dosage regimens for treating or preventing DCM or HF may comprise flat dosing (i.e., administering the same dose repeatedly at pre-determined intervals) or comprise a loading dose (i.e., administrating an initial dose that is higher or different than subsequent, serial doses). For the purposes of either type of dosing regimen an effective dose may be administered topically, parenterally, subcutaneously, subdermally, intradermally, or intramuscularly. In preferred embodiments, administration comprises subcutaneous injection.

In some embodiments, a loading dose and the subsequent serial doses may be administered via the same route (e.g., subcutaneously), while in some embodiments, a loading dose and the subsequent serial doses may be administered via different routes (e.g., parenterally and subcutaneously, respectively). In some embodiments, the loading dose is administered as a single injection. In some embodiments, the loading dose is administered as multiple injections, which may be administered at the same time or spaced apart at defined intervals. The subsequent serial doses of a loading dose regimen are generally lower than the loading dose.

In some embodiments of the disclosed methods and uses, the duration of treatment or prevention is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based on the appearance of symptoms of DCM or HF. In preferred embodiments, duration of treatment or prevention is about 12 weeks to about 24 weeks, about 12 to about 36 weeks, about 12 to about 48 weeks, or about 24 to about 36 weeks.

The present disclosure provides uses of a therapy described herein in the manufacture of a medicament for the treatment or prevention of DCM or HF, for normalizing expression of CD36 in subjects with or at risk for DCM or HF, and/or for normalizing heart function, such as by normalizing left ventricle function (e.g. left ventricle ejection fraction). All of the disclosed doses, dosing regimens, routes of administrations, biomarkers, and therapeutic endpoints are applicable to these uses as well.

Routes and frequency of administration of the therapeutic agents disclosed herein, as well as dosage, will vary from individual to individual as well as with the selected drug, and can be readily established using standard techniques. In general, the pharmaceutical compositions can be administered, by injection (e.g., intracutaneous, intramuscular, intravenous or subcutaneous), intranasally (e.g., by aspiration) or orally.

A “solid oral dosage form,” “oral dosage form,” “unit dose form,” “dosage form for oral administration,” and the like are used interchangeably, and refer to a pharmaceutical composition in the form of a tablet, capsule, caplet, gelcap, geltab, pill and the like.

Dosage forms typically include an “excipient,” which as used herein, is any component of a dosage form that is not an API. Excipients include binders, lubricants, diluents, disintegrants, coatings, barrier layer components, glidants, and other components. Excipients are known in the art (see HANDBOOK OF PHARMACEUTICAL EXCIPIENTS, FIFTH EDITION, 2005, edited by Rowe et al., McGraw Hill). Some excipients serve multiple functions or are so-called high functionality excipients. For example, talc can act as a lubricant, and an anti-adherent, and a glidant. See Pifferi et al., 2005, “Quality and functionality of excipients” Farmaco. 54:1-14; and Zeleznik and Renak, Business Briefing: Pharmagenerics 2004.

A suitable dose is an amount of a compound that, when administered as described above, is capable of promoting an anti-cardiac disease therapeutic response. Such response can be monitored using conventional methods. In general, for pharmaceutical compositions, the amount of each drug present in a dose ranges from about 100 g to 5 mg per kg of host, but those skilled in the art will appreciate that specific doses depend on the drug to be administered and are not necessarily limited to this general range. Likewise, suitable volumes for each administration will vary with the size of the patient.

In the context of treatment, a “therapeutically effective amount” of a drug is an amount of or its pharmaceutically acceptable salt which eliminates, alleviates, or provides relief of the symptoms for which it is administered. The disclosed compositions are administered in any suitable manner, often with pharmaceutically acceptable carriers. Suitable methods of administering treatment in the context of the present invention to a subject are available, and, although more than one route can be used to administer a particular composition, a particular route can often provide a more immediate and more effective reaction than another route. The dose administered to a patient, in the context of the present invention, should be sufficient to effect a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

In general, an appropriate dosage and treatment regimen involves administration of the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### Methods of Prognosis

Methods of treating, predicting, or preventing cardiovascular disorders are also provided herein and can be used in combination with the diagnostic and prognostic method provided for detection and monitoring of the CD36 mutations described herein (e.g. Y325X). In some embodiments, methods for detecting CD36 mutations are provided for treating, predicting, diagnosing, providing prognosis, determining the likelihood that a patient will respond to a therapy, and/or managing a disease or condition related to a cardiovascular disorder. In some embodiments, methods for detecting CD36 mutations are provided for treating or preventing a disease or condition related to a CD36 mutation, in conjunction with one or more therapies effective in treating the disease or condition, including but not limited to drug therapy.

Provided herein, in certain embodiments, are methods for predicting the onset of DCM or HF and/or determining the prognosis of a patient having a cardiovascular disease (e.g., DCM or HF), comprising: performing a nucleic acid detection assay on a sample comprising CD36 nucleic acid from a patient to determine whether the nucleic acid comprises a mutation, preferably wherein the mutation is loss-of-function mutation or a nonsense (stop-gain) mutation; and diagnosing the patient as having a poor prognosis when the mutation is detected.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis can be expressed as the amount of time a patient can be expected to survive. Alternatively, a prognosis can refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis can be expressed as the number of years, on average that a patient can expect to survive as a result of a condition or disease. The prognosis of a patient can be considered as an expression of relativism, with many factors effecting the ultimate outcome. For example, for patients with certain conditions, prognosis can be appropriately expressed as the likelihood that a condition can be treatable or curable, or the likelihood that a disease will go into remission, whereas for patients with more severe conditions prognosis can be more appropriately expressed as likelihood of survival for a specified period of time. In certain embodiments, a prognosis can be expressed as the likelihood that a subject or patient who is positive for a mutation in a particular gene, but who does not present with a particular disease (e.g., a disease linked to or associated with the gene), will subsequently develop the particular disease.

The term “poor prognosis” as used herein, in the context of a patient having a cardiovascular disease and a mutation in the CD36 gene, refers to an increased likelihood that the patient will have a worse outcome in a clinical condition relative to a patient diagnosed as having the same disease but without the mutation. A poor prognosis can be expressed in any relevant prognostic terms and can include, for example, the expectation of a reduced duration of remission, reduced survival rate, and reduced survival duration.

Provided herein is a method for prognosing a subject having or suspected of having dilated cardiomyopathy (DCM) or heart failure (HF), comprising: (a) detecting in a sample obtained from the subject an expression level of full-length CD36 protein; and (b) prognosing the subject as having a poor prognosis if the expression level of CD36 protein is less than a reference level, wherein the reference level is the corresponding level of expression of CD36 protein in a sample obtained from a subject not having or not suspected of having DCM or HF.

Provided herein, in certain embodiments, are methods for determining the likelihood that a patient will respond to a therapy. In some embodiments, provided is a method for determining whether a subject having or suspected of having dilated cardiomyopathy (DCM) or heart failure (HF) is likely to respond to a therapy for DCM or HF, comprising: (a) detecting in a sample obtained from the subject an expression level of full-length CD36 protein; and (b) determining that the subject is less likely to respond to the therapy if the expression level of CD36 protein is less than a reference level when compared to a subject whose CD36 protein expression level is not less than the reference level, wherein the reference level is the corresponding level of expression of CD36 protein in a sample obtained from a subject not having or not suspected of having DCM or HF.

### VI. Kits

The present disclosure also contemplates detection, diagnostic, prognostic, and treatment systems in kit form. In some embodiments, a kit can be used for conducting the diagnostic, prognostic, or treatment methods described herein. In some embodiments, a kit can be used as a companion diagnostic for detection of a CD36 mutation in a subject has received one or more treatments for a cardiovascular disease or condition (e.g. DCM or HF).

Typically, the kit should contain, in a carrier or compartmentalized container, reagents useful in any of the above-described embodiments of the methods.

A detection system provided herein can include a kit which contains, in an amount sufficient for at least one assay, any of the hybridization assay probes and amplification primers for detection of CD36 wild type and mutant nucleic acids, and/or antibodies against CD36 wild-type and mutant proteins in a packaging material. In some embodiments the kit further comprises reagents for the detection of additional mutations in CD36.

In some embodiments, the kit includes one or more primers or probes suitable for amplification and/or sequencing. The primers can be labeled with a detectable marker such as radioactive isotopes, or fluorescence markers.

In some embodiments, the kit may also include primers for the amplification of one or more housekeeping genes. Non-limiting examples of housekeeping genes include GAPDH, ACTB, TUBB, UBQ, PGK, and RPL.

Typically, the kits will also include instructions recorded in a tangible form (e.g., contained on paper or an electronic medium) for using the packaged probes, primers, and/or antibodies in a detection assay for determining the presence or amount of mutant nucleic acid or protein in a test sample.

The various components of the detection, diagnostic, prognostic, and treatment systems can be provided in a variety of forms. For example, the required enzymes, the nucleotide triphosphates, the probes, primers, and/or antibodies can be provided as a lyophilized reagent. These lyophilized reagents can be pre-mixed before lyophilization so that when reconstituted they form a complete mixture with the proper ratio of each of the components ready for use in the assay. In addition, the systems of the present inventions can contain a reconstitution reagent for reconstituting the lyophilized reagents of the kit. In preferred kits, the enzymes, nucleotide triphosphates and required cofactors for the enzymes are provided as a single lyophilized reagent that, when reconstituted, forms a proper reagent for use in the present amplification methods.

In some embodiments, the kit includes suitable buffers, reagents for isolating nucleic acid, and instructions for use. Kits can also include a microarray that contains nucleic acid or peptide probes for the detection of the mutant genes or encoded proteins, respectively.

In some embodiments, the kits can further contain a solid support for anchoring the nucleic acid or proteins of interest on the solid support. In some embodiments, the target nucleic acid can be anchored to the solid support directly or indirectly through a capture probe anchored to the solid support and capable of hybridizing to the nucleic acid of interest. Examples of such solid supports include, but are not limited to, beads, microparticles (for example, gold and other nanoparticles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, antigen/antibody.

Exemplary packaging for the kit can include, for example, a container or support, in the form of, e.g., bag, box, tube, rack, and is optionally compartmentalized. The packaging can define an enclosed confinement for safety purposes during shipment and storage.

Provided herein is a kit for prognosing dilated cardiomyopathy (DCM) or heart failure (HF) in a patient diagnosed with DCM or HF comprising: (i) at least one PCR primer pair for PCR amplification of a CD36 gene or at least one probe for hybridizing to a CD36 gene under stringent hybridization conditions; and (ii) at least one PCR primer pair for PCR amplification of at least one housekeeping gene.

### EXAMPLES

These examples are provided for illustrative purposes only and not to limit the scope of the claims provided herein.

### Example 1. Materials and Methods

The present example describes materials and methods used in the study described herein.

### Study Populations

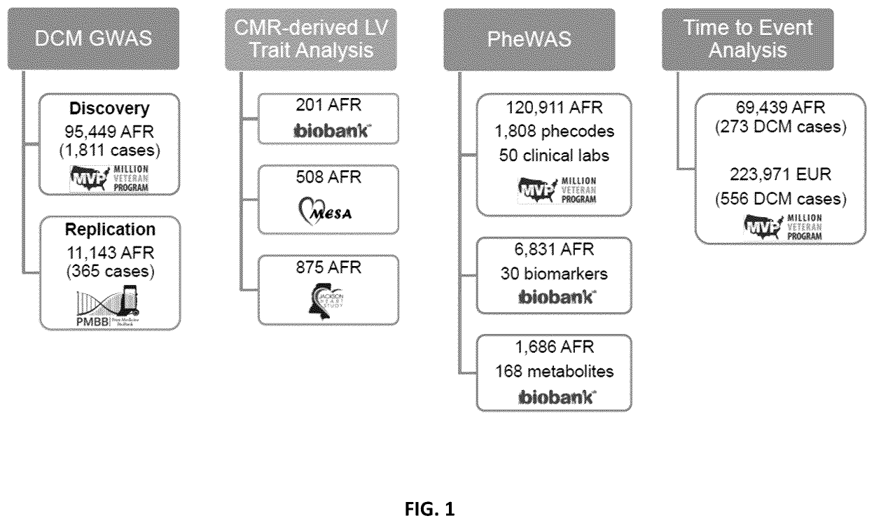

Study participants were evaluated from MVP to compare the prevalence of DCM among African (AFR) and European (EUR) ancestry participants. For primary genetic association analyses, 95,449 AFR participants (1,811 DCM cases) from MVP and pursued replication of genetic association results in 11,143 AFR participants (365 DCM cases) from the Penn Medicine Biobank (PMBB) were studied. Genetic association results using cardiac magnetic resonance imaging (CMR) data from UK Biobank (UKB), the Multi-Ethnic Study of Atherosclerosis (MESA), and the Jackson Heart Study (JHS) were replicated. A phenome-wide association scan (PheWAS) was conducted in MVP and UK Biobank, and observational, time-to-event analyses were performed in participants from MVP (FIG. 1). All studies were approved by their respective IRB committees and all participants provided informed consent.

### Clinical Phenotypes

Disease phenotypes in MVP and PMBB were defined using hospital billing code data. The primary outcome phenotype was DCM, defined by the presence of International Classification of Diseases, 10th Revision (ICD-10) billing code 142.0 (“Dilated cardiomyopathy”). Secondarily, we evaluated genetic associations with the broader clinical phenotype of heart failure with reduced ejection fraction (HFrEF) in MVP. Controls excluded other forms of cardiomyopathy and heart failure. Clinical cardiovascular risk factors in MVP—hypertension, body mass index (BMI), diabetes, atrial fibrillation, chronic kidney disease, total cholesterol, high-density lipoprotein (HDL) cholesterol, smoking status, and alcohol intake—were derived from the medical record.

### Genotyping, Imputation, Ancestry Definition

Genotyping and quality control in MVP have been reported previously.

The harmonized race/ethnicity and genetic ancestry (HARE) approach, developed by MVP, was used to assign individuals to populations or groups. This machine learning algorithm leverages information from both the self-identified race/ethnicity data from the survey and data from the genome-wide array to create respective variables for downstream analyses. HARE categorized Veterans into four mutually exclusive groups: (1) non-Hispanic White, (2) non-Hispanic Black, (3) Hispanic or Latino, or (4) Asian.

ADMIXTURE9 was used to calculate genetic loadings on five 1000Genomes reference populations representing the majority of ancestry within the United States—GBR (British), PEL (Peruvian), YRI (Yoruba/Nigerian), CHB (Han Chinese), and LWK (Luhya/Kenyan). Population-specific principal components (PCs) were computed using EIGENSOFT v.6.

Detailed information on the genetic methods can be found in the genotyping and imputation methods for PMBB, UKB, MESA, and JHS.

### Genome-Wide Association Analysis and Replication

A genome-wide association analysis (GWAS) for DCM was performed in AFR participants from MVP using logistic regression with adjustment for age at study enrollment, biological sex, and the first 10 population-specific PCs in plink2a. The association of any lead signal with HFrEF in MVP was further examined, before pursuing replication in AFR participants from PMBB using the same logistic regression model and covariates.

Additional replication was performed by testing the association of any lead variant with cardiac MRI-derived measures of left ventricular (LV) structure and function (left ventricular ejection fraction [LVEF], and body surface area-indexed values of left ventricular end-diastolic volume [LVEDVi], left ventricular end-systolic volume [LVESVi], and left ventricular mass [LVMi]) in UKB, MESA, and JHS study participants without documentation of clinical heart failure, cardiomyopathy, or coronary artery disease. Linear regression models were used adjusting for age, sex, and the first 10 PCs, and meta-analysis across studies was performed using fixed-effects and inverse-variance weighting.

### Gene Prioritization

Publicly available transcriptomic (from the Genotype-Tissue Expression (GTEx) Version 8) and proteomic (from the Atherosclerosis Risk in Communities [ARIC] study) data were used to identify a likely causal gene mediating the uncovered genetic signal. Statistical fine-mapping was combined with assessments of CD36 expression in the left ventricle and CD36 levels in plasma to identify the common variant likely mediating the genetic association.

### Assessment of Functional Variant Across Ancestral Groups

To investigate the role of ancestry in the significant SNP associations, logistic regression was undertaken within AFR MVP participants for DCM, adjusting for genetic loadings from ADMIXTURE (see above) and the functional variant.

### Phenome Wide Association Study (PheWAS)

A phenome-wide association study of the functional variant was performed with 1,808 ICD code-based phenotypes and 49 clinical laboratory biomarkers in AFR individuals of MVP (N=120,911), as well as 30 laboratory biomarkers (N=6,831) and 168 metabolites (N=1,686) in AFR individuals of UKB. Logistic regression was used for the analysis of disease phenotypes, and linear regression for analyses of continuous biomarkers and metabolites, each adjusting for age at enrollment, biological sex, and the first 10 PCs in an additive model. Significance was defined as 2.77×10⁻⁰⁵ (0.05/1,808) for the binary trait analysis and 2.02×10⁻⁰⁴ (0.05/247) for continuous traits.

### Incident-Disease Analysis

In AFR and EUR MVP participants free of baseline DCM, heart failure or coronary artery disease, time-to-event analyses were pursued to determine the risk of incident DCM. Such analyses were conducted to estimate: (1) the ancestry-specific population attributable fraction (PAF) for DCM of the functional variant as compared to relevant clinical risk factors; (2) the hazards of DCM in African as compared to European ancestry participants, and the extent to which adjustment for clinical risk factors and/or the functional genetic variant attenuate the observed risk difference.

### Example 2. Baseline Participant Characteristics

The present example describes characteristics of participants of the study described herein. Of the 95,449 MVP participants of African ancestry (AFR), the mean age was 56.5 years, the majority were male (84.9%), and most had hypertension (69.9%). The prevalence of DCM was nearly two-fold among individuals of AFR (1.9%) versus European (EUR) ancestry (1.0%, p<0.001) (Table 2). Of note, AFR participants experienced a higher prevalence of chronic kidney disease and diabetes, but EUR participants had a higher rates of atrial fibrillation and coronary artery disease.

TABLE 2
Baseline characteristics of VA Million Veteran
Program Participants by ancestry group.

| | | |
|---|---|---|
| | EUR | AFR |
| Clinical Characteristic | (N = 370,085) | (N = 95,449) |

| | | | | |
|---|---|---|---|---|
| Age | 62.8 | (13.9) | 56.5 | (12.3) |
| Male | 340,312 | (92.0) | 81,059 | (84.9) |
| Hypertension | 234,027 | (63.2) | 66,746 | (69.9) |
| Body mass index | 29.3 | (5.6) | 30.0 | (5.9) |
| Diabetes | 77,043 | (20.8) | 25,725 | (27.0) |
| Previous CAD | 83,268 | (22.5) | 15,546 | (16.3) |
| Atrial Fibrillation | 25,801 | (7.0) | 2,806 | (2.9) |
| Chronic Kidney Disease | 36,432 | (9.8) | 13,771 | (14.4) |
| Total Cholesterol | 174.2 | (40.9) | 178.3 | (41.1) |
| HDL Cholesterol | 46.1 | (13.9) | 50.4 | (15.4) |
| Current Drinker | 204,513 | (55.3) | 49,340 | (51.7) |
| Ever Smoker | 268,662 | (72.6) | 64,306 | (67.4) |
| DCM cases | 3,786 | (1.0) | 1,811 | (1.9) |

Data are shown in N (%) or mean (SD).
Alcohol intake was defined by the AUDIT-C questionnaire.
CAD = Coronary Artery Disease, HDL = high density cholesterol, DCM = dilated cardiomyopathy. AFR = non- Hispanic Black, EUR = non-Hispanic White.

### Example 3. GWAS and Gene Prioritization

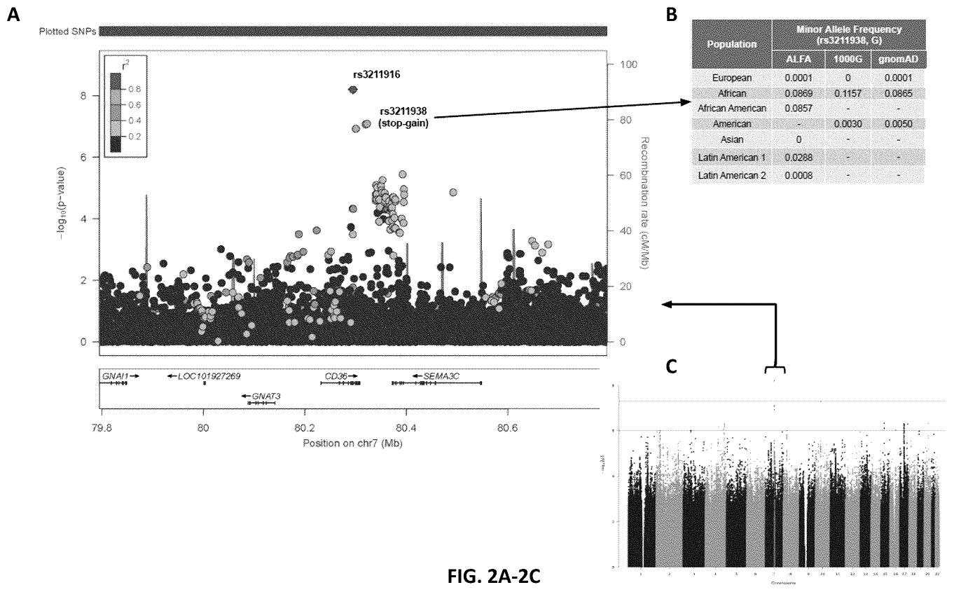

The present example describes a genome-wide association study analysis of DCM. Genetic association analyses of DCM in MVP AFR participants (N=1,811 cases) yielded one locus at chromosome 7 that reached genome-wide significance (FIG. 2); the minor allele [C] of the lead variant rs3211916 (within the CD36 gene) was associated with increased risk of DCM (OR=1.34 [1.21-1.48], P=6.22×10₋₉). Adjustment for the lead variant did not show any other signals in the region. The association of the lead variant with DCM was robust to adjustment for cardiovascular risk factors Table 3).

TABLE 3
Association of rs3211916 and rs3211938 with dilated cardiomyopathy
(DCM) in VA Million Veteran Program (MVP) after adjustment
for cardiovascular risk factors.

| | Association between | Association between |
|---|---|---|
| | rs3211916 and DCM | rs3211938 and DCM |
| Covariates | OR (95% CI) | OR (95% CI) |
| Age, sex, and PC 1-5 | 1.34 (1.21 to 1.48) | 1.33 (1.19 to 1.48) |

Further adjustment for

| | | |
|---|---|---|
| Hypertension | 1.33 (1.21 to 1.47) | 1.32 (1.19 to 1.47) |
| BMI | 1.34 (1.21 to 1.48) | 1.34 (1.20 to 1.49) |
| Diabetes | 1.34 (1.22 to 1.49) | 1.34 (1.20 to 1.49) |
| Previous CAD | 1.33 (1.21 to 1.48) | 1.32 (1.18 to 1.47) |
| Atrial Fibrillation | 1.37 (1.24 to 1.52) | 1.35 (1.21 to 1.51) |
| Chronic Kidney Disease | 1.34 (1.21 to 1.48) | 1.32 (1.18 to 1.47) |
| Total Cholesterol | 1.33 (1.20 to 1.47) | 1.33 (1.19 to 1.48) |
| HDL Cholesterol | 1.36 (1.23 to 1.51) | 1.32 (1.18 to 1.47) |
| Smoking status | 1.34 (1.21 to 1.48) | 1.33 (1.19 to 1.48) |
| Alcohol intake | 1.34 (1.21 to 1.48) | 1.34 (1.20 to 1.49) |
| All CVD risk factors | 1.32 (1.19 to 1.48) | 1.31 (1.17 to 1.47) |

Each association was adjusted for age, sex the first five principal components and the risk factor listed using logistic regression with an additive model.
DCM = dilated cardiomyopathy.
PC = principal component.

Out of 17 common genetic loci previously linked to DCM in GWAS of predominantly EUR ancestry participants, 16 had a directionally-consistent association with DCM in our AFR GWAS in MVP, of which 10 achieved nominal significance (p<0.05)

rs3211916 was associated (P<5×10⁻⁰⁴) with expression of CD36 in several tissues including left ventricle and atrial appendage, and not associated with expression of genes beyond CD36 in GTEx (Table 4), limiting the likelihood that expression of genes beyond CD36 underlies the association between this signal and DCM. Additionally, the signal for DCM at chromosome 7 colocalized with regional summary statistics for CD36 gene expression in the left ventricle tissue in GTEx (PPH4=0.94), and plasma CD36 levels in the ARIC study (PPH4=0.98). In aggregate, this strongly implicates CD36 as the likely causal gene mediating the risk of DCM at the locus.

TABLE 4
Associations between C allele at rs3211916 and
gene expression from GTEx Version 8 portal.

| | Gene | | | |
|---|---|---|---|---|
| Gencode Id | Symbol | Tissue | NES | P-Value |
| ENSG00000135218.17 | CD36 | Heart - Left | −1.30 | 1.00E−22 |
| | | Ventricle | | |
| | | Heart - Atrial | −1.00 | 4.90E−13 |
| | | Appendage | | |
| | | Whole Blood | −0.72 | 4.40E−12 |
| | | Muscle - Skeletal | −0.75 | 1.80E−10 |
| | | Adipose - | −0.63 | 3.40E−09 |
| | | Subcutaneous | | |
| | | Skin - Not | −0.76 | 1.10E−07 |
| | | Sun Exposed | | |
| | | (Suprapubic) | | |
| | | Nerve - Tibial | −0.79 | 7.40E−07 |
| | | Lung | −1.10 | 4.10E−06 |
| | | Artery - Tibial | −0.59 | 1.30E−05 |
| | | Thyroid | −0.79 | 2.70E−05 |
| | | Stomach | −0.86 | 3.80E−05 |
| | | Esophagus - Mucosa | −0.77 | 1.30E−04 |

Associations with P < 5 × 10⁻⁴ are reported (https://gtexportal.org/home/snp/rs3211916)

Statistical fine mapping at the locus yielded a 95% credible set comprising four common variants, including the lead signal rs3211916. Notably, left ventricular CD36 expression and plasma CD36 data revealed rs3211938 as the peak variant in the region. The G allele at rs3211938 is a nonsense (stop-gain) mutation in linkage disequilibrium (LD) with the lead variant, rs3211916 (r²=0.64 in 1000G African ancestry, r²=0.65 in MVP AFR), and confers increased risk of DCM (OR=1.33 [1.20-1.48], P=1.18×10⁻⁷), which was also robust to cardiovascular risk factor adjustment (Table 3). Conditioning on the lead variant abrogated the association for rs3211938, indicating that the two polymorphisms represent the same association signal for DCM. Due to its functional consequences, high LD with the lead variant, and strong effect estimate, rs3211938 was considered the likely causal variant at the region (hereby referred to as the “functional variant”) and served as the focus of downstream analyses.

### Example 4. Assessment of Functional Variants Across Ancestral Groups

The present example illustrates the assessment of functional variants across ancestral groups (i.e. AFR or EUR ancestry). The risk allele (G) at rs3211938 was specific to AFR ancestry, with frequency approximately 9% in African ancestry populations, but less than 0.01% in European ancestry populations, a consistent observation across four study populations and various populations in dbSNP (FIG. 2).

### Example 5. Replication of Functional Variant Association Signal

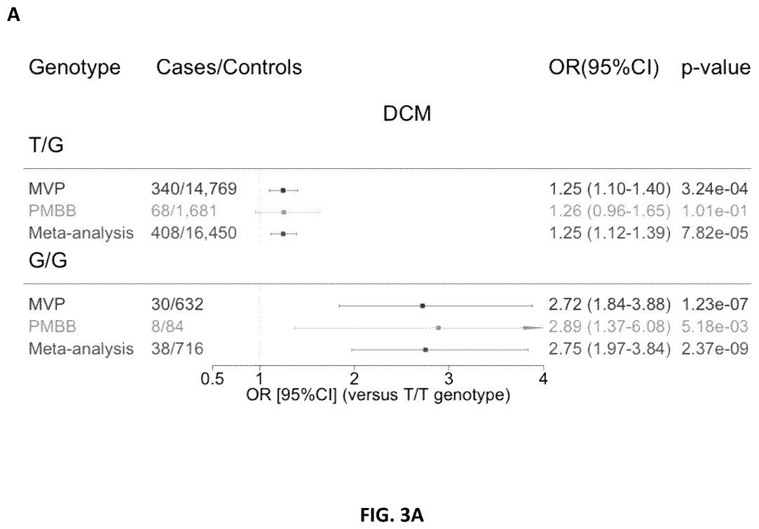

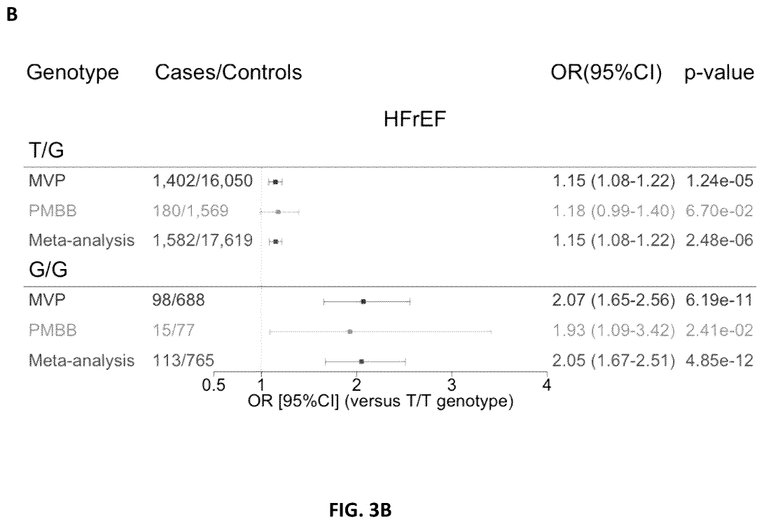

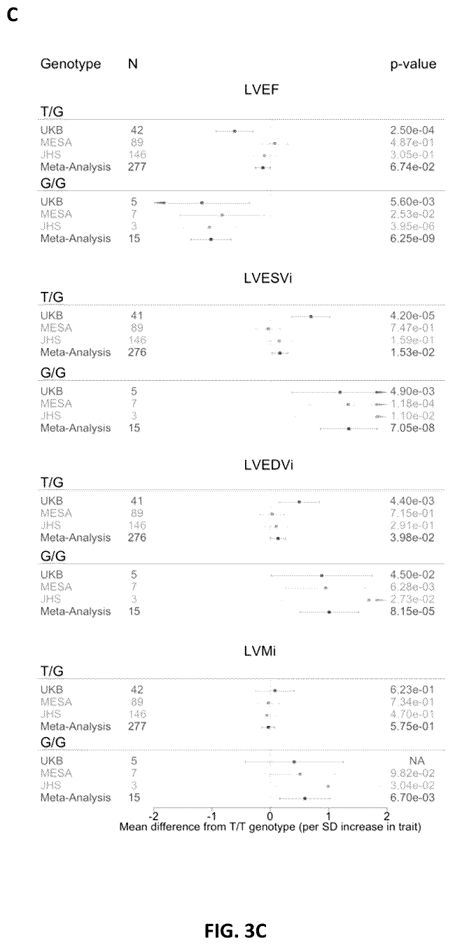

The present example illustrates the replication of the association signal at rs3211938 by assessing a range of phenotypes indicative of left ventricular dysfunction in the African ancestry subsets of four independent cohorts. First, the associations of rs3211938 with DCM and HFrEF in PMBB were examined. Using an additive model, the association of rs3211938 with DCM per G-allele in PMBB (OR=1.37) was consistent with MVP (OR=1.33), with a meta-analyzed OR of 1.33 (P=5.22×10⁻⁹). However, analyses by genotype were suggestive of a non-linear association between the number of G alleles at rs3211938 and DCM, which was consistent across MVP and PMBB. As compared to those with the T/T genotype, heterozygotes for the risk allele (T/G) had an OR of 1.25 for DCM (Pₘₑₜₐ=7.82×10⁻⁵) while homozygotes for the risk allele (G/G) had an OR of 2.75 (Pₘₑₜₐ=2.37×10⁻⁹, FIG. 3A). Associations of rs3211938 with HFrEF were also consistent across MVP and PMBB and suggestive of a non-linear shape; risk allele heterozygotes (T/G) had an OR of 1.13 (Pₘₑₜₐ1.70×10⁻⁴) while risk allele homozygotes (G/G) had an OR of 1.96 (Pₘₑₜₐ=1.74×10⁻⁹, FIG. 3B).

Next, using CMR data from UKB, MESA, and JHS, the association of rs3211938 with subclinical measures of LV structure and function was tested. In each of the three study populations, risk allele homozygosity (G/G) was associated with approximately 1 standard deviation decreases in LVEF and increases in LVESVi and LVEDVi, and more modest increases in LVMi, changes most consistent with a subclinical DCM phenotype (P<0.01 for all, FIG. 3C). For LVEF, this corresponded to a 6.8%, 6.3%, and 9.4% decrease in baseline values across UK Biobank, MESA, and JHS, respectively, for individuals with the G/G versus T/T genotype.

### Example 6. Phenome-Wide Association Scan

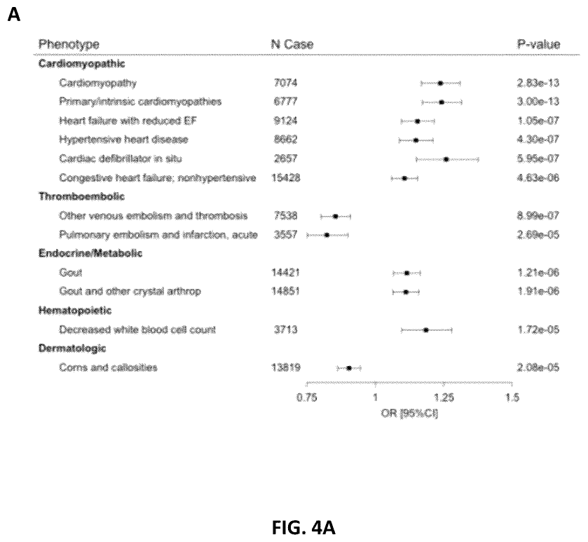

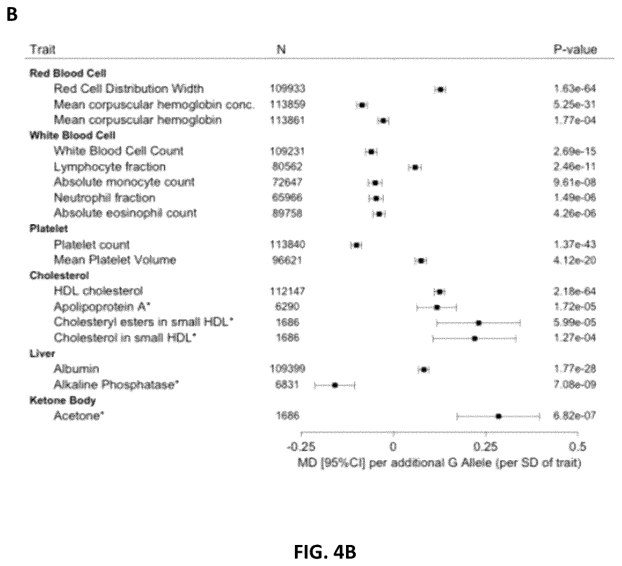

To determine the broad phenotypic consequences of CD36 perturbation, the association of rs3211938 with a spectrum of cardiovascular and non-cardiovascular outcomes and traits in AFR participants from MVP and UKB was tested. Consistent with the above link to LV dysfunction, the functional variant was directly associated with several phenotypic codes of relevance to cardiomyopathy and heart failure, such as those for “primary/intrinsic cardiomyopathy,” “hypertensive heart disease,” and placement of “cardiac defibrillator in situ,” the latter likely a marker of severe LV dysfunction. In addition, there were direct associations with metabolic outcomes such as “gout,” and inverse associations with thromboembolic outcomes such as “pulmonary embolism” (FIG. 4A). Significant associations with HDL-cholesterol-related traits were observed, a number of blood cell and platelet traits, and increased levels of serum acetone (FIG. 4B).

### Example 7. Population-Level Impact of rs3211938 on DCM

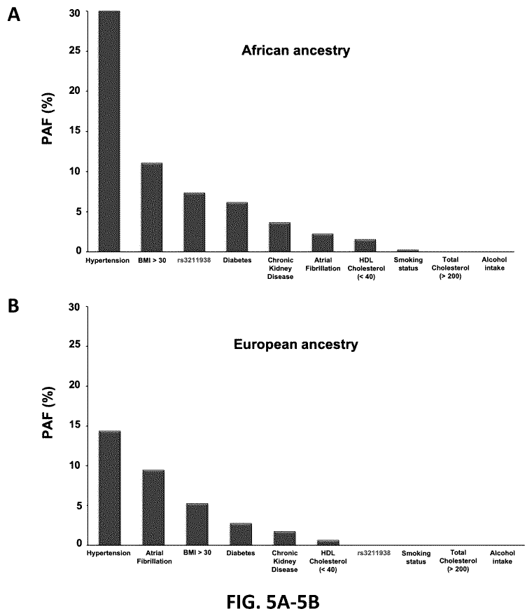

To assess the population-wide impact of rs3211938 on risk for DCM, cases of DCM incident to study enrollment were examined. Over a mean of 4.21 person-years, 829 incident DCM cases occurred, with rates of 59.3 and 91.4 per 100,000 person-years for MVP AFR and EUR individuals, respectively. The population attributable fraction (PAF) of DCM due to cardiovascular risk factors as compared to rs3211938 was first estimated. In AFR participants, the highest PAF for DCM was for hypertension (32.1%, P=1.78×10₋₃) followed by BMI (13.5%, P=0.02). The PAF for rs3211938 alone was 9.0% (P=3.37×10₋₃), which was greater than that for other individual clinical risk factors including diabetes, chronic kidney disease, atrial fibrillation, lipids, smoking, and alcohol intake. In EUR participants, the PAF for DCM for rs3211938 was considered to be 0%, due to the extreme rarity of the variant (FIG. 5; Table 5).

TABLE 5
Population attributable fraction (PAF) for cardiovascular risk factors
and rs3211938 in AFR and EUR ancestry individuals for DCM.

| | AFR ancestry | EUR ancestry |
|---|---|---|
| Risk Factors | PAF (%) | PAF (%) |
| Hypertension | 31.2 | 14.4 |
| BMI (>30) | 11.1 | 5.3 |
| Diabetes | 6.2 | 2.8 |
| Atrial Fibrillation | 2.3 | 9.5 |
| Chronic Kidney Disease | 3.7 | 1.8 |
| Total Cholesterol (>200) | −0.6 | −1.8 |
| HDL Cholesterol (>40) | 1.6 | 0.7 |
| Smoking status | 0.3 | 0.0 |
| Alcohol intake | −2.9 | −3.3 |
| rs3211938 | 7.4 | 0* |

*The PAF estimate for an exceedingly rare exposure is assumed to be 0.

The extent to which differences in DCM risk between AFR and EUR populations are accounted for by differences in cardiovascular risk factors versus the presence of rs3211938 was assessed. In an age-and-sex-adjusted model, AFR ancestry was associated with a 79% (HR: 1.79, 95% CI: 1.54-2.07, P<0.001) increase in DCM risk compared to EUR ancestry. Adjustment for rs3211938 attenuated the association between AFR HARE group and DCM (HR: 1.63, 95% CI: 1.39-1.92) to the same extent as adjustment for all cardiovascular risk factors combined (HR: 1.68, 95% CI: 1.44-1.96); adjustment for rs3211938 and cardiovascular risk factors further attenuated this association (HR: 1.53, 95% CI: 1.39-1.77, Table 6).

TABLE 6
Incident Dilated Cardiomyopathy hazard ratios
by group after adjusting for other risk factors

| | HR (95% CI) for the association | |
|---|---|---|
| | between AFR ancestry (versus | |
| Adjustment | EUR ancestry) and incident DCM | |
| Age and sex only | | |
| EUR | 1 | (ref) |
| AFR | 1.79 | (1.54-2.07) |
| Age, sex, and all | | |
| cardiovascular | | |
| risk factors | | |
| EUR | 1 | (ref) |
| AFR | 1.68 | (1.44-1.96) |
| Age, sex, and rs3211938 | | |
| EUR | 1 | (ref) |
| AFR | 1.63 | (1.39-1.92) |
| Age, sex, rs3211938, | | |
| and all cardiovascular | | |
| risk factors | | |
| EUR | 1 | (ref) |
| AFR | 1.53 | (1.35-1.83) |

Associations between AFR ancestry (versus EUR ancestry) and DCM adjusting for rs3211938, cardiovascular risk factors, or both. Cardiovascular risk factors include hypertension, body mass index (BMI), diabetes, previous coronary artery disease (CAD), atrial fibrillation, chronic kidney disease, total cholesterol, high density lipoprotein (HDL) cholesterol, smoking status and alcohol intake.

### Example 8. Silencing of CD36 Reduces Lipid Uptake and Impairs Mitochondrial Function in iPSC-Derived Cardiomyocytes (iPSC-CMs)

The present Example illustrates the relationship between CD36, lipid uptake, and mitochondrial function in cardiomyocytes, and demonstrates a biological function of CD36 in heart cell function.

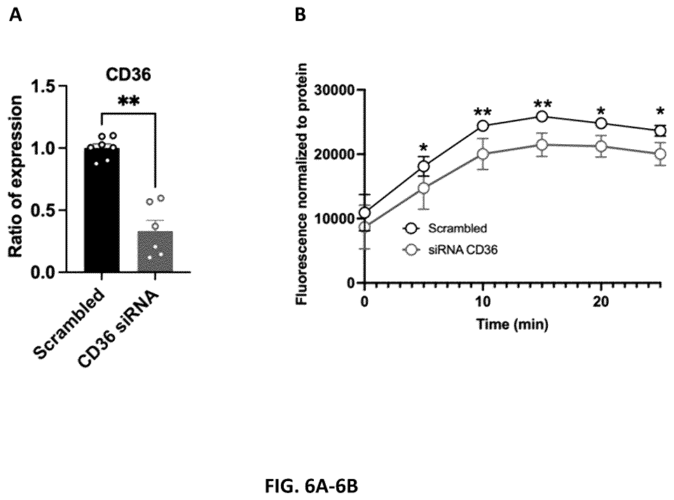

Briefly, an in vitro knock-down model was used to assess the cellular consequences of reduced CD36 activity in live, cultured induced pluripotent stem cell (iPSC)-derived cardiomyocytes (iPSC-CMs). iPSC-CMs were treated with short interfering RNAs (siRNAs) targeting CD36. Following treatment, iPSC-CMs were harvested, and CD36 expression was measured by polymerase chain reaction (PCR). As shown in FIG. 6A, CD36 expression was successfully reduced by CD36 siRNA treatment.

Next, physiological consequences of CD36 silencing were assessed in the iPSC-CM model. As shown in FIG. 6B, CD36 knockdown resulted in a reduction in the uptake of lipids into iPSC-CMs, including free fatty acids (FFAs), as measured using a fluorescence-based fatty acid uptake kit (Abcam). These results validated some of the purported cellular deficits associated with CD36 deficiency in cardiomyocytes, including dysregulated energy utilization.

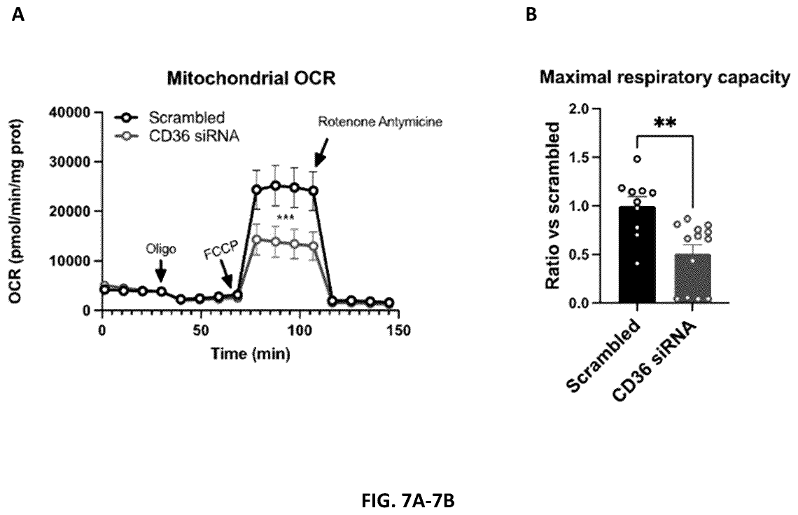

Since fatty acids can be used as a key energy source, it was hypothesized that CD36-deficient iPSC-CMs would exhibit deficits in mitochondrial function. iPSC-CMs treated with siRNAs targeting CD36 were assessed for deficits in mitochondrial function. As shown in FIG. 7A, CD36-deficient iPSC-CMs exhibited a significant reduction in the carbonyl cyanide-p-trifluoromethoxyphenylhydrazone (FCCP)-induced mitochondrial oxygen consumption rate (OCR), relative to iPSC-CMs treated with a control (scrambled) siRNA. In addition, as shown in FIG. 7B, CD36-deficient iPSC-CMs exhibited a marked reduction in maximal respiratory capacity when compared to control iPSC-CMs, as measured using a Seahorse metabolic analyzer (Agilent). It was conjectured that the observed impairments in mitochondrial function in CD36-deficient iPSC-CMs were due to decreased lipid uptake, since reduction in lipid uptake is known to cause a shift in mitochondrial energy utilization to less-efficient fuel sources. Accordingly, these data suggest that targeting mitochondrial energetics may have therapeutic potential for patients with CD36 deficiency.

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of detecting CD36 expression or function in a sample, comprising, consisting of, or consisting essentially of
(a) obtaining a sample from a subject having, suspected of having, or at risk for dilated cardiomyopathy (DCM) or heart failure (HF); and
(b) detecting (i) an expression level of full-length CD36 protein in the sample, or (ii) the presence or absence of a nucleic acid encoding a CD36 protein that comprises a mutation that results in a loss of function of the CD36 protein encoded by the nucleic acid.

2. The method of claim 1, wherein detecting comprises sequencing the nucleic acid encoding the CD36 protein.

3. The method of claim 1, wherein detecting comprises amplifying the nucleic acid encoding the CD36 protein.

4. (canceled)

5. (canceled)

6. A method for determining whether a subject having, suspected of having, or at risk for dilated cardiomyopathy (DCM) or heart failure (HF) is likely to respond to a therapy for DCM or HF, comprising:
(a) detecting in a sample obtained from the subject an expression level of full-length CD36 protein; and
(b) determining that the subject is less likely to respond to the therapy if the expression level of CD36 protein is less than a reference level when compared to a subject whose CD36 protein expression level is not less than the reference level,
wherein the reference level is the corresponding level of expression of CD36 protein in a sample obtained from a subject not having or not suspected of having DCM or HF.

7. The method of claim 6, wherein HF is heart failure with reduced ejection fraction (HFrEF).

8. A method for determining whether a subject having, suspected of having, or at risk for dilated cardiomyopathy (DCM) or heart failure (HF) is likely to respond to a therapy for DCM or HF, comprising:
(a) analyzing a biological sample obtained from the subject, wherein the biological sample comprises a CD36 protein or a nucleic acid encoding a CD36 protein in the sample;
(b) detecting the presence or absence of a CD36 mutation that results in loss-of-function of CD36; and
(c) determining that the subject is less likely to respond to the therapy if the mutation is detected.

9. The method of claim 8, wherein HF is heart failure with reduced ejection fraction (HFrEF).

10. The method of claim 8, wherein the mutation is a stop-gain variant.

11. The method of claim 8, wherein the mutation is Y325X relative to SEQ ID NO:1.

12. The method of claim 8, wherein the therapy is selected from the group consisting of salt restriction, ACE inhibitors, diuretics, beta blockers, anticoagulants, coenzyme Q, angiotensin receptor blockers (ARBs), aldosterone antagonists, and sodium glucose cotransporter-2 (SGLT-2) inhibitors.

13. A method for treating dilated cardiomyopathy (DCM) or heart failure (HF) in a subject, the method comprising:
(a) analyzing a biological sample obtained from the subject, wherein the biological sample comprises a CD36 protein or a nucleic acid encoding a CD36 protein in the sample;
(b) detecting the presence or absence of a CD36 mutation that results in loss-of-function of CD36; and
(c) administering to the subject a pharmacological agent targeting myocardial energetics if the mutation is detected.

14. The method of claim 13, wherein HF is heart failure with reduced ejection fraction (HFrEF).

15. The method of claim 13, wherein the mutation is a stop-gain variant.

16. The method of claim 13, wherein the mutation is Y325X relative to SEQ ID NO:1.

17. The method of claim 13, further comprising (d) administering to the subject a pharmacological agent selected from the group consisting of salt restriction, ACE inhibitors, diuretics, beta blockers, anticoagulants, and coenzyme Q, angiotensin receptor blockers (ARBs), aldosterone antagonists, and sodium glucose cotransporter-2 (SGLT-2) inhibitors, if the mutation is not detected.

18. A method for treating dilated cardiomyopathy (DCM) or heart failure (HF) in a subject, the method comprising:
(a) analyzing a biological sample obtained from the subject, wherein the biological sample comprises a CD36 protein or a nucleic acid encoding a CD36 protein in the sample;
(b) detecting in the biological sample an expression level of full-length CD36 protein; and
(c) administering to the subject a pharmacological agent targeting myocardial energetics if the expression level of CD36 is less than a reference level,
wherein the reference level is the corresponding level of expression of CD36 protein in a sample obtained from a subject not having or not suspected of having DCM or HF.

19. The method of claim 18, wherein HF is heart failure with reduced ejection fraction (HFrEF).

20. The method of claim 18, further comprising (e) administering to the subject a pharmacological agent selected from the group consisting of salt restriction, ACE inhibitors, diuretics, beta blockers, anticoagulants, and coenzyme Q, angiotensin receptor blockers (ARBs), aldosterone antagonists, and sodium glucose cotransporter-2 (SGLT-2) inhibitors, if the expression level of CD36 is not less than the reference level.

21. The method of claim 13, wherein the pharmacological agent is a gene therapy.

22.-26. (canceled)
